(19) United States
(12) Patent Application Publication
(10) Pub. No.: US 2005/0289667 A1
(43) Pub. Date: Dec. 29, 2005

(54) Biological gene transfer system for eukaryotic cells

(75) Inventor: Richard A. Jefferson, Canberra (AU)

Correspondence Address: CAROL NOTTENBURG, 814 32ND AVE 5, SEATTLE, WA, 98144, US

(73) Assignee: CAMBIA

(21) Appl. No.: 10/953,392

(22) Filed: Sep. 28, 2004

Related U.S. Application Data
(63) Continuation of application No. 60/583,426, filed on Jun. 28, 2004.

(51) Int. Cl. A01H 1/00; C12N 15/82

(52) U.S. Cl. 800/279; 800/294

(57) ABSTRACT

This invention relates generally to technologies for the transfer of nucleic acids molecules to eukaryotic cells. In particular non-pathogenic species of bacteria that interact with plant cells are used to transfer nucleic acid sequences. The bacteria for transforming plants usually contain binary vectors, such as a plasmid with a vir region of a Ti plasmid and a plasmid with a T region containing a DNA sequence of interest.

### CROSS-RELATED APPLICATION

This application claims the benefit of U.S. Provisional Application No. 60/583,426, filed 28 Jun. 2004, which is incorporated by reference in its entirety.

### REFERENCE TO SEQUENCE LISTING ON COMPACT DISK

The sequence listing of this application is provided separately in a file named “414B seq List.txt” on one (1) compact disc. The content of this file, which was created on 28 Sep. 2004 and is 30,956 bytes, is incorporated in its entirety.

### BACKGROUND OF THE INVENTION

This invention relates generally to technologies for the transfer of nucleic acids molecules to eukaryotic cells and in particular technologies using non-pathogenic bacteria to transfer nucleic acid sequences to eukaryotic cells, e.g. to plant cells.

There are three essential processes for commercial use of transformation technology in crops: (i) introduction of new DNA into appropriate plant cells/organs; (ii) growth or multiplication of successfully transformed cells/plants, often involving selection or discrimination methodologies; and (iii) expression of transgene(s) in target cells/organs/stages.

Each of these processes is represented by several alternative technologies of varying quality and efficiencies. The first step, however, is the most critical, not only for plants but for transformation of any eukaryotic organism and cell type. There are currently two classes of DNA introduction methods widely used to generate transgenic organisms, physical methods and biological methods.

Physical methods for introducing DNA include particle bombardment, electroporation and direct DNA uptake by or injection into protoplasts. These methods—in their currently practiced forms—have substantial drawbacks. The structure of the introduced DNAs tends to be complex and difficult to control, and the stresses associated with the introduction or the types of regeneration necessary to use these methods are often mutagenic. Furthermore, the patent landscape around these methods varies dramatically, but none are unencumbered.

Biological transformation currently focuses on the use of the natural genetic engineer, Agrobacterium tumefaciens, to transfer defined new DNA sequences into plants. Agrobacterium tumefaciens is a common soil bacterium that naturally inserts some of its genes into plants and uses the machinery of plants to express those genes in the form of compounds that the bacterium uses as nutrients. In the process, some of the transferred genes also cause the formation of plant tumors commonly seen near the junction of the root and the stem, deriving from it the name of crown gall disease. The disease afflicts a great range of dicotyledonous plants (dicots), which constitute one of the major groups of flowering plants. So-called disarmed strains of Agrobacterium are used for plant transformation, which have lost the capacity to form tumors and display a reduced pathogenesis phenotype on plants. There are though at least seven chromosomal virulence genes and several other genes that affect virulence that are still present in commonly employed Agrobacterium strains.

Despite this disadvantage, Agrobacterium-mediated transformation of plants has been widely used for transformation of plant cells. Other shortcomings of using Agrobacterium include a limited host range, and it can only infect a limited number of cell types in that range. Of particular importance, whereas Agrobacterium can infect many dicots, monocotyledonous plants (monocots) are more resistant to infection. Monocotyledonous plants (monocots) however, constitute most of the important food crops in the world (e.g., rice, corn). Monocots are only able to be transformed by Agrobacterium under special conditions and using a special type of cell, the callus cells or other dedifferentiated tissue (e.g., U.S. Pat. No. 5,591,616; No. 6,037,552; No. 5,187,073; No. 6,074,877). Nonetheless, some monocots and some dicots, e.g. soybean and other leguminous plants, are still notoriously difficult to transform with Agrobacterium. There also exist huge differences in transformation efficiency between varieties of a given plant species, with some being completely recalcitrant to gene transfer by Agrobacterium.

Despite these drawbacks of Agrobacterium, other bacteria systems have not been developed for transformation of eukaryotic cells. Other bacteria genera were not believed to be suitable for transforming plants. Indeed, Agrobacterium is widely known as the only bacterial genus that has the capacity for trans-kingdom gene transfer. While some reports allegedly demonstrated that the tumor-inducing ability of Agrobacterium could be transferred to other related genera, including rhizobia (Klein and Klein, Arch Microbiol. 52:325-344, 1953; Kern, Arch. Microbiol. 52:325-344, 1965), the results were not uniformly repeatable nor was there any physical proof of gene transfer. For example, Hooykaas, Schilperoort and their colleagues in the mid to late 70's reported that some bacterial species, Rhizobium trifolii and R. leguminosarum in particular, were capable of tumor formation on plants after introduction of a Ti plasmid from a virulent Agrobacterium (Hooykaas et al., Gen. Microbiol. 98:477-484, 1977; Hooykaas et al., Gen. Microbiol. 4:661-666, 1984), while other species, in particular Rhizobium meliloti (now called Sinorhizobium meliloti), were not (van Veen et al., Plant-Microbe Interactions 1:231-234, 1989). Since then, very little additional work has been done, either to validate that gene transfer occurred or to further examine the ability, if any, of rhizobia to mediate gene transfer. Only very recently has a root-inducing Ri plasmid been found in environmental isolates of Ochrobactrium, Rhizobium, and Sinorhizobium from root mat-infected cucumber and tomatoes (Weller et al., Appl. and Environ. Microbiol 70:2779-2785, 2004), indicating that these bacteria can maintain an Agrobacterium rhizogenes Ri plasmid. No causal relationship with the disease was shown however, nor was there any evidence of DNA transfer to the plants. In addition, Sinorhizobium spp. was shown to be a reservoir of a Ti plasmid, but no tests were done on the functionality of the Ti plasmid in this bacterium (Teyssier-Cuvelle et al. Molec. Ecoli. 8: 1273-1284, 1999). Thus, researchers have essentially only used a single species of Agrobacterium, A. tumefaciens, which was known to successfully transform plant cells.

### BRIEF SUMMARY OF THE INVENTION

Within one aspect of the present invention, a system for transforming eukaryotic cells is provided. In particular, one such system comprises transformation competent bacteria that are non-pathogenic for plants and contain a first nucleic acid molecule comprising genes required for transfer and a second nucleic acid molecule comprising one or more sequences that enable transfer of a DNA sequence of interest. In various embodiments, the genes required for transfer are vir genes of a Ti plasmid from Agrobacterium or homologues of vir genes, such as tra genes from plasmids like RK2 or RK4. In other embodiments, the sequence enabling transfer is a T-border sequence of a Ti plasmid from Agrobacterium. In certain embodiments, the DNA sequence of interest is located between two T-border sequences. In other embodiments, the sequence enabling transfer is an oriT sequence from any mobilizable bacterial plasmid.

In another aspect, the bacteria contain a first plasmid comprising a vir gene region of a Ti plasmid, such as a disarmed Ti plasmid from Agrobacterium, and a second plasmid comprising one or more T-border or oriT sequences and a DNA sequence of interest. In yet another aspect, the bacteria contain a single plasmid comprising a vir gene region of a Ti plasmid and one or more T-border or oriT sequences operatively linked to a DNA sequence of interest.

The plasmids and nucleic acid molecules are designed to transfer DNA sequences of interest to eukaryotic cells. In one embodiment, the plasmid that is introduced in the bacteria to induce the transfer of the DNA sequences of interest to the eukaryotic cells may be the Ti plasmid of A. tumefaciens, or a derivative thereof, containing all or at least part of the vir genes. The plasmid generally does not contain a T-DNA region. In some cases, the vir genes are inducible, in other cases, the vir genes are constitutively expressed. In one embodiment, the plasmid has one or more virG sequences. In another embodiment, the helper plasmid has a broad-host range origin of replication, such as the origin of replication from RK2 plasmid. In other embodiments, the helper vector has one or more oriT sequences, such as the oriT from RP4. In some embodiments, the vector has a selectable marker.

The second nucleic acid molecule or plasmid can be a T-DNA plasmid or T-DNA-like plasmid, which has sequences that serve the same function as T-DNA borders. In certain embodiments, the homologue of T-DNA border sequence is an origin of transfer (oriT). When the second plasmid is a T-DNA plasmid, it has at least one T-DNA border sequence.

The sequences that enable transfer (e.g., T-border sequences) of a DNA sequence of interest are operatively linked to the DNA sequence of interest, such that the DNA sequence of interest is transferred to the recipient eukaryotic cell. Moreover, the nucleic acid molecules may contain genes encoding selectable products to allow selection in the bacteria or in the eukaryotic cell.

The non-pathogenic bacteria that interact with plants or plant cells are obtained and transfected with the above nucleic acid molecules or plasmids by conjugation, electroporation, or other means. Suitable bacteria include, but are not limited to, non-pathogenic Rhizobium, Sinorhizobium, Mesorhizobium, Bradyrhizobium, Pseudomonas, Azospirillum, Rhodococcus, Phyllobacterium, Xanthomonas, Burkholderia, Erwinia, and Bacillus.

The bacteria containing these plasmids are contacted with suitably prepared plants, plant cells, or plant tissues for a time sufficient to allow transfer of the DNA sequence of interest to the cells. In one embodiment, the plant or cells or tissue that is transformed is selected for. When plant cells or tissues are used, the transformed cells are regenerated into a plant.

These and other aspects of the present invention will become evident upon reference to the following detailed description and attached drawings. In addition, various references are set forth below which describe in more detail certain procedures or compositions (e.g., plasmids, etc.), and are therefore incorporated by reference in their entirety.

### BRIEF DESCRIPTION OF THE DRAWINGS

FIG. 1 provides the current taxonomical hierarchy of bacteria in the Rhizobiales order.

FIG. 2 displays a map of exemplary binary vectors.

FIG. 3 shows partial nucleotide sequences of 16S rDNA, atpD and recA genes for Rhizobium spp. NGR234 (streptomycin-resistant strain ANU240) (SEQ ID NOS:1-3), Sinorhizobium meliloti 1021 (SEQ ID NOS:4-6), Mesorhizobium loti MAFF303099 (SEQ ID NOS:7-9), Phyllobacterium myrsinacearum Cambia isolate WB 1 (SEQ ID NOS:10-11), Bradyrhizobium japonicum USDA110 (SEQ ID NOS:12-14), and Agrobacterium tumefaciens EHA105 (SEQ ID NOS:15-17).

FIG. 4 is a picture of an electrophoresis gel containing amplification products of DNA from 2-2000 Agrobacterium EHA 101 cells that are diluted into a culture of 2×10⁴ Rhizobium leguminosarum cells. The upper band is amplified R. leguminosarum 16SrDNA, and the lower band is amplified A. tumefaciens 16SrDNA. Lane 1, 2000 Agrobacterium cells; Lane 2, 200 Agrobacterium cells; Lane 3, 20 Agrobacterium cells; Lane 4, 2 Agrobacterium cells; Lane 5, Agrobacterium cells only; Lane 6, 100 bp molecular DNA ladder (400-1000 bp).

FIG. 5 shows the results of an amplification analysis of transformants of Ti plasmid-cured LBA288 cells electroporated with Ti plasmid DNA isolated from EHA101. The following primers were used: lane a, Atu16S (SEQ ID NOS:21-22); lane b, attScirc (SEQ ID NOS:23-24); lane c, attSpAT (SEQ ID NOS:25-26); lane d, AtuvirG (SEQ ID NOS:27-28); lane e, nptI (SEQ ID NO:29-30); lane f, virB (SEQ ID NOS:31-32). LBA288, Ti plasmid-cured Agrobacterium strain; EHA101, donor strain for Ti plasmid DNA; transformant 1 and 2, independent transformants of LBA288.

FIG. 6 illustrates a strategy for integration of the oriT from RP4 in the Ti plasmid of EHA105, utilizing a suicide vector (pWBE58) harboring a homologous sequence of the Ti plasmid (virG).

FIG. 7 is a Southern blot analysis on genomic DNA from two A. tumefaciens Ti plasmid::suicide vector integrants showing duplication of the virG region (EHA105 pTi1) and the accA region (EHA105 pTi2) respectively.

FIG. 8 shows a vector map for binary vector pCAMBIA1105.1. BGUS, gusplus™ (U.S. Pat. No. 6,391,547) gene; HYG(R), hygromycin resistance gene; MCS, multi-cloning site.

FIG. 9 shows a vector map for binary vector pCAMBIA1105.1R. BGUS, gusplus™ gene (U.S. Pat. No. 6,391,547); HYG(R), hygromycin resistance gene; MCS, multi-cloning site (note that the MCS differs from the one in pCAMBIA1105.1.

FIG. 10 is an electrophoresis gel showing the result of amplification analysis on DNA from a strain of Rhizobium spp. NGR234 (upper panel) and a strain of S. meliloti 1021 (middle panel), harboring the A. tumefaciens modified Ti plasmids pTi1 and pTi3 respectively, and the binary vector pCAMBIA1105.1R. The following primers were used: lane a, Sme16SrDNA (SEQ ID NOS:33-34); lane b, NodD1NGR234 (SEQ ID NOS:35-36); lane c, SmeNodQ+NodQ2 (SEQ ID NOS:37-39); lane d, VirB (SEQ ID NOS:31-32); lane e, VirB11FW2+M13REV (identifies pTi1; SEQ ID NOS:40-41); lane f, M13FW+MoaAREV2 (identifies pTi3; SEQ ID NOS:42-43); lane g, HygR510 (SEQ ID NOS:44-45); lane h and h′, 1405.1FW+M13FW (SEQ ID NOS:46+42; identifies the specific MCS in the binary vector; positive control in lane h is pCAMBIA1105.1R, and in h′, pCAMBIA1105.1); lane i, Atu16SrDNA (SEQ ID NOS:21-22); lane j, attScirc (SEQ ID NOS:23-24); lane k, attSpAT (SEQ ID NOS:25-26); lane M, combined 100 bp and 1 kb DNA ladder

FIG. 11 provides images of rice calli stained for GUS (β-glucuronidase) activity (arrows point to some of the blue regions) following co-cultivation with A. tumefaciens, S. meliloti and Rhizobium spp. respectively, each harboring a Ti plasmid and binary vector.

FIG. 12 provides images of tobacco leaf discs stained for GUS activity following co-cultivation with A. tumefaciens, S. meliloti and Rhizobium spp. respectively, each harboring a Ti plasmid and binary vector; arrows point to some of the blue GUS regions.

FIG. 13 shows Arabidopsis seedlings germinating on hygromycin-containing medium following floral dip transformation with Rhizobium spp. NGR234 harboring pTi1 and pCAMBA1105.1R; the arrow points to a growing, hygromycin-resistant seedling.

FIG. 14 shows GUS stained leaf tips from regenerated tobacco shoots following co-cultivation with gene transfer competent strains of A. tumefaciens, and S. meliloti respectively.

FIG. 15 provides amplification data for the HygR gene using primers Hyg700 (SEQ ID NOS:82-83) (upper panel) and MCS (SEQ ID NOS:46 and 79) (lower panel) on tobacco shoots (genotype Wisconsin38) regenerated following co-cultivation with gene transfer competent S. meliloti (2-1, 6, 7-1, 11-1) and A. tumefaciens (1, 2, 3) respectively.

FIG. 16 provides a picture of rooted tobacco shoots regenerated after co-cultivation with S. meliloti harboring pTi3 and pC1105.1R.

FIG. 17 provides images of A. Sinorhizobium meliloti-mediated, genetically transformed rice calli with GUS activity (blue) and non-transformed rice calli (white) and B. Sinorhizobium meliloti-mediated, genetically transformed rice shoot with GUS activity (blue) visible in the roots, callus at the base of developing shoot and in the tip of the shoot.

FIG. 18 provides Southern blots for four independent tobacco plants (2-2; 3-2; 6; 13) transformed by S. meliloi containing pTi3 and pC1105.1R. Left panel, hygromycin probe; Right panel, the same blot that has been stripped and probed with GUSplus. (+), single copy transformed rice plant; BV, binary vector pC1105.1R equivalent to one genome copy.

### DETAILED DESCRIPTION OF THE INVENTION

As noted above, the present invention provides bacterial species that are useful for transforming eukaryotic cells, especially plant cells. Bacterial species useful in this invention are bacteria that can interact with plants and that are non-pathogenic. The bacteria are made gene transfer competent by transfection with a nucleic acid molecule, such as a Ti helper plasmid from Agrobacterium or a derivative thereof, comprising all or part of the vir gene region or functional equivalents, and a second nucleic acid molecule or plasmid that comprises a DNA sequence of interest operatively linked to one or more sequences enabling transfer of the sequence of interest to the eukaryotic plant cell. In certain aspect the bacteria are made gene transfer competent by transfection with a single nucleic acid molecule that comprises the vir genes or homologues and the DNA sequence of interest operatively linked to the sequence(s) enabling transfer.

Identification of Suitable Non-Pathogenic Bacteria

The bacteria for use in this invention are those that can interact with plants, without being harmful for the plant or plant cells, i.e. they are non-pathogenic. Non-pathogenic bacteria are those that are benign or beneficial to plants. Non-pathogenic bacteria are those that do not cause a disease state. Symptoms of a disease state include death of cells of plant tissues that are invaded, progressive invasion of vascular elements and necrosis of adjacent tissues, maceration of tissues (e.g., soft-rot), and abnormal cell division. (For more information on plant pathogenic bacteria, see “Kado, C I, “Plant Pathogenic Bacteria” in M. Dworkin et al., eds., The Prokaryotes: An Evolving Electronic Resource for the Microbiological Community, 2nd edition, release 3.0, 21 May 1999, Springer-Verlag, New York, http://link.springer-ny.com/link/service/books/10125/.) Some advantages of using non-pathogenic bacteria include an increased quality of transformation and ease of use, minimal or no necrosis or browning, and lack of a hypersensitive necrosis response. Moreover, the bacteria of this invention may interact efficiently with other plant species than Agrobacterium does, offering huge opportunities for exploitation of diverse well-evolved bacteria-plant interactions and convert them into gene transfer systems. These bacteria hence offer valuable alternatives to choose from when planning transformation experiments for a given eukaryotic species, particularly if it is a species that is known to be difficult to transform using Agrobacterium.

The bacteria for use in this invention interact with plant tissues. While root-associating bacteria, rhizobia, are probably best known, the bacteria useful in this invention may associate with any plant tissue, such as roots, leaves, meristems, sexual organs, and stems. Such bacteria include, but are not limited to, species of Sinorhizobium, Mesorhizobium, Bradyrhizobium, Pseudomonas, Azospirillum, Rhodococcus, Phyllobacterium, Xanthomonas, Burkholderia, Ochrobacter, Erwinia, and Bacillus.

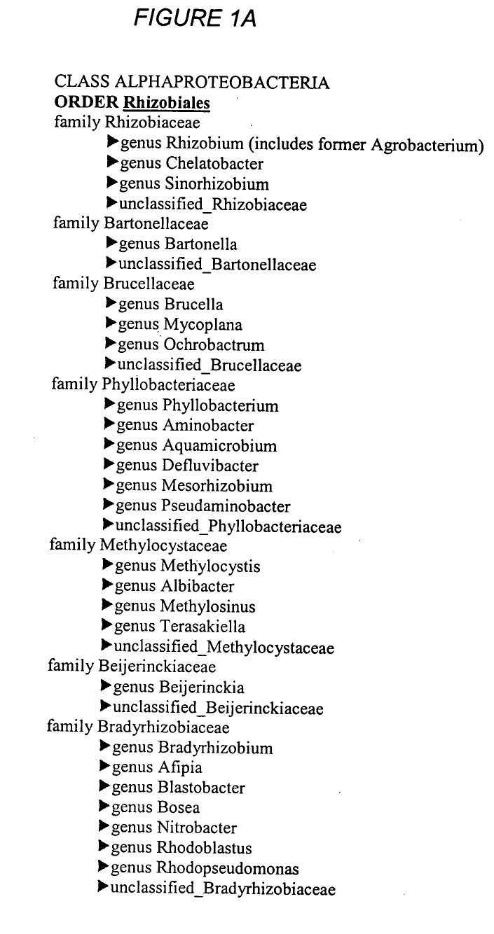

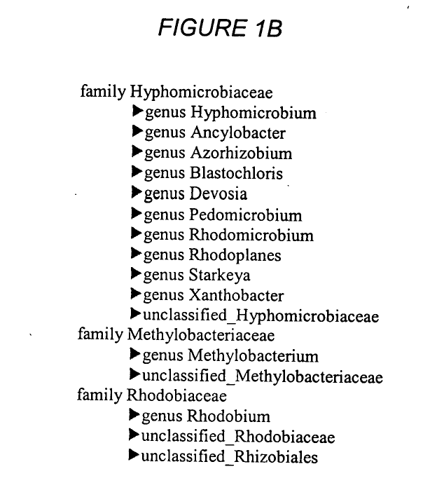

One of the well known non-pathogenic class of bacteria that are plant-associated include rhizobia, bacteria that fix nitrogen. Rhizobia comprise a group of Gram negative bacteria, which have the ability to produce nodules on roots or, in some cases, on stems of leguminous plants (e.g., beans, peas, lentils, and peanuts). Currently there are several genera of rhizobia distinguished and nearly 40 species, some of which are presented in FIG. 1. These genera represent different families within subgroup 2 of the α-Proteobacteria (Gaunt et al., IJSEM 51:2037-2048, 2001). This includes species in the genera Rhizobium, Sinorhizobium, Allorhizobium, Mesorhizobium, Bradyrhizobium, Azorhizobium, Methylobacterium, and others.

Molecular data, such as similarity of rDNA gene sequences, have contributed to the current view of bacterial taxonomy. Given the fluidity of taxonomy as more data are obtained, one of the best methods for identification of bacterial species is identity (similarity) of nucleic acid sequences of 16S rDNA genes; sequences of additional gene loci have confirmed the 16S rDNA-based phylogenies (Gaunt et al., IJSEM 51:2037-2048, 2001). Thus, the names of bacterial genera and species may change over time as taxonomy is revised. For example, by comparison of rDNA genes, Agrobacterium tumefaciens was discovered to be the same species as Rhizobium radiobacter and is now known by that name. “What's in a name? That which we call a rose/By any other word would smell as sweet.” (William Shakespeare, Romeo and Juliet, act 2, sc. 1, 1.75-8 1599).

Bacteria can be obtained from soil samples, plant tissues, germplasm banks, strain collections, and commercial sources. Conditions for culturing different bacteria are well known. The bacteria can be screened for antibiotic sensitivities to find a suitable antibiotic that allows growth under selective conditions that prevent the growth of other bacteria. Antibiotic resistances and sensitivities are determined by plating the test bacteria on solid medium containing different concentrations of antibiotics and counting the number of colonies. Alternatively, the rate of growth in the presence of different antibiotics and different concentrations can be determined by assaying the number of bacteria in the medium at time intervals. Numbers of bacteria and growth curves are readily determined by plating on permissive solid medium and counting colonies or by spectrophotometric absorbance measurements.

The species of the bacteria of this invention are conveniently determined by molecular techniques. An accepted method in the art is comparison of rDNA sequence obtained from the bacteria to rDNA sequences determined from known bacteria genera or species, although other gene sequences can be used instead of or in addition to rDNA sequences. In the Examples, the bacteria employed in this invention are identified by comparisons of 16S rDNA, recA, and atpD nucleotide sequences to a database of sequences; all of these gene sequences have been used previously for phylogenetic studies in bacteria (Gaunt et al., IJSEM 51:2-37-2048, 2001). The sequences are generally obtained by sequencing of amplified fragments of genomic DNA. Consensus primers for amplification of these genes and many others can be found in the literature (e.g. (Tan et al., Appl. Environm. Microbiol 8:1273-1284, 2001); (Gaunt et al., IJSEM 51:2037-2048, 2001)) or can be designed based on the alignment of sequences from related species. Preferably the match between sequences is at least 90%, at least 95%, or at least 99%.

For the convenience of rapidly confirming the strain or strains used in this invention, bacterial species may also be identified by amplification using species-specific or genus-specific primer sequences. These may include primers that specifically amplify at least part of the 16S rDNA region, other chromosomal regions, and plasmid-born sequences. Primers are tested against a broad collection of bacterial strains (e.g., those used in the lab), and only those that amplify the correct product from the expected species, and not from the other species, are used in subsequent identification assessments.

In one aspect of this invention, the bacteria used for gene transfer should be capable of obtaining and maintaining a plasmid. In some embodiments, the plasmid is a functional Ti plasmid or at least part of a Ti plasmid. As part of a study to control crown gall disease in plants caused by Agrobacterium, Teyssier-Cuvelle et al. (Molec. Ecoli. 8:1273-1284, 1999) investigated soil microflora for bacteria that could obtain and maintain a Ti plasmid through conjugation from Agrobacterium cells. The taxonomy of the transconjugant bacteria was determined by amplification of rDNA genes and comparison with a database of rDNA gene sequences. The authors identified two new bacterial ssp., closely related to Sinorhizobium and Rhizobium, which are used in the Examples. The Ti plasmid obtained and maintained by the bacteria of this invention may be modified in order to increase its uptake or stability or both in certain species. For example, the Ti plasmid can be modified by insertion of a replication origin that is recognized in these bacteria species, or an origin of transfer (oriT) that make the plasmid mobilizable, or by removal or mutation of genes that are either not essential for gene transfer or of which the removal or mutation improves the stability of the Ti plasmid or its mobilization to other bacteria.

The bacteria should also be capable of inducing or constitutively expressing the genes that are involved in transfer of the DNA sequence of interest. These genes are the virulence genes encoded by the vir operons or homologues of the virulence genes, such as the tra genes. When vir genes are used, induction is generally achieved through the action of phenolic compounds that are naturally released by wounded plant cells or compounds, e.g. acetosyringone, which are added to the medium in which the bacteria are growing before explant infection. Any means to show that the vir genes, tra genes or other homologues are expressed can be used to establish functionality. Exemplary means include Western blot analysis of the proteins using specific antibodies, analysis of expression of a reporter gene linked to the promoter of any of the genes (e.g. employing a vir promoter-lacZ fusion), or microscopic visualization of the cellular localization of the proteins (e.g. virD4 or virE2), that are fused to a reporter gene such as green fluorescent protein. Alternatively, the formation of a single stranded transfer intermediate, such as a T-DNA molecule, can be directly visualized, such as on a Southern blot with undigested genomic DNA following acetosyringone induction of bacterial cultures.

The bacteria that are found to maintain a first nucleic acid molecule, such as a disarmed Ti plasmid, should be capable of expressing the genes that are involved in transfer of DNA sequences of interest to plant cells. In one embodiment, the DNA sequences of interest are provided on a T-DNA plasmid on which these genes are flanked by one or two T-DNA borders. The T-DNA borders are the sites of nicking of the T-DNA plasmid by the virD2 protein, leading to the formation of the relaxosome (T-complex), which is then transferred to the plant cell through the virB transmembrane complex.

In another embodiment, the DNA sequences of interest are provided on a plasmid that has no T-DNA borders, but instead contains one or two sequences that serve the same function as T-DNA borders, i.e. sites for nicking and excision of the single stranded DNA region containing the DNA sequences of interest (Waters et al., Proc. Natl. Acad. Sci. USA 88:1456-1460, 1991; Ward et al., Proc. Natl. Acad. Sci. USA 88:9350-9354, 1991). These nicking sites can be composed of the origin of transfer regions (oriT) of plasmids such as RSF1010 or CloDF13, both of which have been shown to be transported by the virB transmembrane complex (Buchanan-Wollastan et al., 1987; Escudero et al., 2003). As for the T-DNA borders, there may be one or more oriT regions. If two oriT regions are present, one oriT region will generally be located at either side of the DNA sequence of interest. A procedure for the transfer of DNA sequences of interest from Agrobacterium cells to plant and yeast cells using non-T-DNA, mobilizable vectors has been described in WO 2001/064925 A1 (Escudero et al., Mol. Microbiol 47:891-901, 2003). The vector was derived from the limited host-range plasmid CloDF13, which contains the oriT and mobB and mobC genes from CloDF13 and a plant expression cassette containing the GUS gene, and was mobilized to plant cells by recruitment of the virulence apparatus of Agrobacterium. Transformed plant tissues were shown to express GUS activity.

In yet another embodiment, the bacteria for use in this invention are capable of maintaining the Agrobacterium Ti plasmid transfer genes, encoded by the virB operon, and possibly other vir genes, on a broad-host range plasmid that is not a complete Ti plasmid. In addition, they are capable of maintaining a second mobilizable plasmid that contains the gene(s) of interest to be transferred to plant cells, e.g. a derivative of CloDF13 as is used in WO 2001/064925.

In addition, the bacteria of this invention attach to plant tissue or make contact to cells in one or another way in order to transfer the DNA of interest to plant cells. For strains not known to attach or interact with plant cells, verification of attachment or contact may be assessed by any number of methods. For example, bacteria can be labeled with fluorescein and incubated with plant tissue; attachment can then be visualized by fluorescence microscopy. Alternatively, the transfer of bacterial proteins involved in T-DNA transfer or integration (e.g. virD2, virE2, virF), or induction of plant genes involved in T-DNA integration (e.g. RAT5) may also be assessed.

Preparation of Nucleic Acid Molecules, Inclduing Plasmids

The bacteria are transfected with nucleic acid molecules, described above. In this section, preparation of the nucleic acid molecules is described in terms of plasmids. For bacteria that contain nucleic acid molecules that are not plasmids (e.g., integrated into the bacterial genome), generally plasmids are used as the starting material.

In one aspect of this invention, two plasmid vectors are employed. The vectors are: (i) a wide-host-range, small replicon, which usually has an origin of replication (oriV) that permits the maintenance of the plasmid in a wide range of bacteria including E. coli and the bacteria of this invention, and (ii) a second plasmid, which, when it is a Ti plasmid, is considered to be “disarmed”, since its tumor-inducing genes located in the T-DNA have been removed. (U.S. Pat. Nos. 4,940,838, 5,149,645 and 5,464,753).

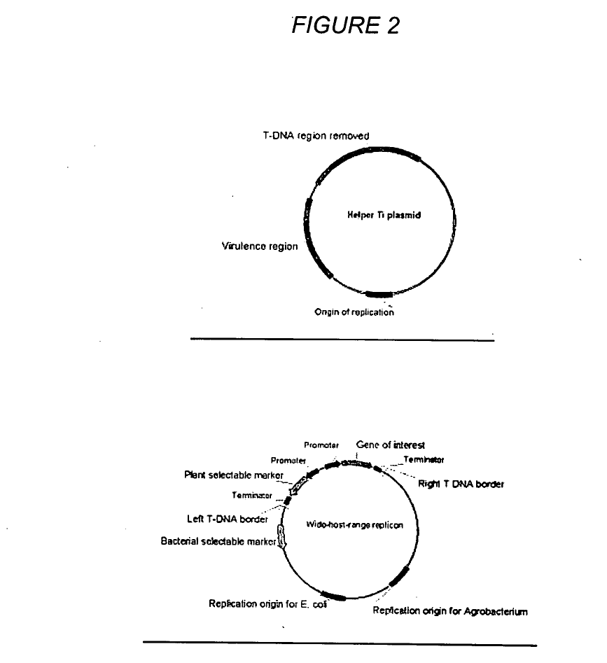

The first plasmid contains the DNA sequence(s) of interest operatively linked with the left and right T-DNA borders (or at least the right T-border). When two border sequences are used, the DNA sequence of interest is located in between the border sequences. When only one border is used, the DNA sequence of interest is located close enough and in a position to be transferred. into the target eukaryotic cells. For expression of the sequence of interest, the sequence is under control of a promoter. A schematic of exemplary plasmids is shown in FIG. 2. In certain embodiments, the plasmid has a sequence that is capable of forming a relaxosome (U.S. 2003/0087439A1). An exemplary mobilizable plasmid is derived from RSF1010 (Scholz et al., Gene 75 (2), 271-288, 1989, GenBank Accession M28829) and CloDF13 (Escudero et al., Mol Microbiol. 47:891-901, 2003; GenBank Accession NC002119).

The second plasmid is typically a broad-host range plasmid, and comprises at least part of the vir genes of the Ti plasmid or homologous genes, such as tra genes. While the entire vir gene or tra gene region (or other functional homologues) is generally used, one or more of the genes may be deleted or replaced by another homologue as long as the remaining genes are sufficient to cause transfer of the DNA sequence of interest. The vector may also contain an oriV and a selectable marker for maintenance in bacteria. When the nucleic acid molecule is integrated into the bacterial chromosome or other self-replicating bacterial DNA molecule, an oriV is not necessary.

Generally, the vector containing the DNA of interest also contains a selectable or a screenable marker for identifying transformants. The marker preferably confers a growth advantage under appropriate conditions. Well known and used selectable markers are drug resistance genes, such as neomycin phosphotransferase, hygromycin phosphotransferase, herbicide resistance genes, and the like. Other selection systems, including genes encoding resistance to other toxic compounds, genes encoding products required for growth of the cells, such as in positive selection, can alternatively be used. Examples of these “positive selection” systems are abundant (see for example, U.S. Pat. No. 5,994,629). Alternatively, a screenable marker may be employed that allows the selection of transformed cells based on a visual phenotype, e.g. β-glucuronidase or green fluorescent protein (GFP) expression. The selectable marker also typically has operably linked regulatory elements necessary for transcription of the genes, e.g., constitutive or inducible promoter and a termination sequence, including a polyadenylation signal sequence. Elements that enhance efficiency of transcription are optionally included.

An exemplary small replicon vector suitable for use in the present invention is based on pCAMBIA1305.2. Other vectors have been described (U.S. Pat. Nos. 4,536,475; 5,733,744; 4,940,838; 5,464,763; 5,501,967; 5,731,179) or may be constructed based on the guidelines presented herein. The pCAMBIA1305.2 plasmid contains a left and right border sequence for integration into a plant host chromosome and also contains a bacterial origin of replication and selectable marker. These border sequences flank two genes. One is a hygromycin resistance gene (hygromycin phosphotransferase or HYG) driven by a double CaMV 35S promoter and using a nopaline synthase polyadenylation site. The second is the β-glucuronidase (GUS) gene (reporter gene) from any of a variety of organisms, such as E. coli, Staphyloccocus, Thermatoga maritima and the like, under control of the CaMV 35S promoter and nopaline synthase polyadenylation site. If appropriate, the CaMV 35S promoter is replaced by a different promoter. Either one of the expression units described above is additionally inserted or is inserted in place of the GUS or HYG gene cassettes.

The Ti plasmid, which contains genes necessary for transferring DNA from Agrobacterium to plant cells, can also replicate in other genera of bacteria. In particular the Ti plasmid can replicate in rhizobia and, moreover, is stable (i.e. is not readily cured from bacteria). Exemplary rhizobia used in the context of this invention include Rhizobium leguminosarum bv. trifolii (former R. trifolii), Rhizobium spp. NGR234, Mesorhizobium loti, Phyllobacterium myrsinacearum, and Sinorhizobium meliloti (former R. meliloti), all of which are capable of supporting and expressing the genes of the Ti plasmid. In one embodiment, the Ti plasmid is modified by the insertion of another replication origin, typically a broad-host range origin of replication such as the RK2 origin of replication, in order to make the Ti plasmid more stable in some bacteria. Thus, when suitably modified and engineered, these bacteria may be used for transferring nucleic acid sequences into eukaryotic cells, and especially into plant cells.

The helper Ti plasmid that is harbored in the bacteria of this invention lacks the entire T-DNA region but contains a vir region. To assist construction of bacterial strains that have both the small replicon plasmid (or the mobilizable plasmid) and the Ti plasmid, the Ti plasmid may contain a selectable marker, compatible origins of replication, and multiple virG sequences. Although the selectable marker can be the same on both plasmids, preferably the markers are different so as to facilitate confirmation that both plasmids are present. The helper plasmid or the small replicon or mobilizable vector can optionally contain at least one additional virG gene, and optionally a modified virG gene. The additional virG gene(s) can be inserted into the Ti plasmid by any of a variety of methods, including the use of transposons and homologous recombination (Kalogeraki and Winans, Gene 188:69-75, 1997). Homologous recombination can be induced by any method, including the use of a suicide plasmid carrying a cloned fragment of the Ti plasmid (e.g. the virG gene), or a stable replicon that is forced to recombine with the Ti plasmid, e.g. by incompatibility. In addition a gene encoding antibiotic resistance can be included on the fragment with virG. Other sequences of the Ti plasmid may similarly be (completely or partly) duplicated or removed, including large regions that tend to be unimportant for the purposes of this application. Optionally an origin of transfer, such as the oriT of RK2/RP4 may be included (Stabb and Ruby, Enzymol. 358:413-426, 2002). This type of transfer origin allows the mobilization of the Ti plasmid to other bacteria, e.g. to rhizobia, with the help of the transfer functions of RK2/RP4 or similar vectors, including derivatives.

An exemplary helper plasmid is pTiBo542 (1). This highly virulent plasmid is also completely sequenced (P. Oger, unpublished data). Disarmed derivatives pEHA101 and pEHA105 have been widely used (Hood et al., J. Bacteriol. 168:1291-1301, 1986; Hood et al., Transgenic Research 2:208-218, 1993). Other helper plasmids include those of LBA4404, the pGA series, pCG series and others (see, Hellens and Mullineaux, A guide to Agrobacterium binary Ti-vectors. Trends Plant Sci. 5: 446-451, 2000).

The construction of co-integrate vectors is well described, for example in U.S. Pat. Nos. 4,693,976, 5,731,179, and EP 116718 B2.

Transfection of Bacteria

In general, the plasmids are transferred via conjugation or through a direct transfer method to the bacteria of this invention. By transferring a suitably disarmed Ti ‘helper’ plasmid from highly transformation-competent Agrobacterium (e.g. pEHA105 from EHA105) and modified gene transfer T-DNA vectors (e.g. pCAMBIA1305.1) (or mobilizable plasmid) to the bacteria of this invention, transformation competent bacteria are generated. These bacteria can be used to transform plants and plant cells.

The first plasmid, e.g., Ti plasmid can be transferred from Agrobacterium (or other rhizobia) containing the Ti plasmid by biological methods, such as conjugation, or by physical methods, such as electroporation or mediated by PEG (polyethylene glycol). When transferring plasmids from Agrobacterium tumefaciens to a chosen bacterial (e.g., rhizobial) strain, the procedure is aided if Agrobacterium has a chromosomal negative selection marker(s), such as auxotrophy or antibiotic sensitivity. Constitutive conjugation ability of the Ti plasmid can be achieved by deletion of accR and/or traM genes on the plasmid (Teyssier-Cuvelle et al., Molec. Ecoli. 8:1273-1284, 1999). Otherwise, induction of conjugation can be achieved by use of specific opines, naturally produced in crown galls, or utilizing a self-transmissible R plasmid (e.g. R772 or RP4) which may (temporarily) form a co-integrate with the Ti plasmid. If the Ti plasmid has been engineered by insertion of a foreign oriT, e.g. the oriT of RP4/RK2, then conjugation from one bacterium to another bacterium can be achieved with the help of bacterial strains, e.g. E. coli, containing compatible transfer functions on a plasmid or on their chromosomes. This may be done in a triparental mating between donor, acceptor and helper strain, or in a biparental mating between a donor containing the transfer genes and an acceptor. Bacteria are transferred to selective medium and putative transconjugants are plated out to isolate single cell colonies. Following transconjugation, the Agrobacterium may be selected against. If the Agrobacterium is sensitive to an antibiotic that the recipient bacteria are resistant to, either naturally resistant or resistant as a result of having the small replicon plasmid, then that antibiotic may be used to select for the recipient bacterial strain. Similarly, if a helper strain was used, it may be selected against by using the same or a different antibiotic to which the recipient bacteria are resistant. They may also be made antibiotic resistant by integration of a foreign gene conferring antibiotic resistance, e.g. mediated by a transposon vector. Similarly, bacteria that have not taken up the Ti plasmid may be eliminated by selection for the Ti plasmid. Generally this selection will be an antibiotic selection as well, but will depend on the selectable markers in the Ti plasmid.

The presence of the Ti plasmid can be verified by any suitable method, although for ease, amplification of the vir genes or any other Ti plasmid sequence is commonly employed. Vir gene expression in the new host can be checked after induction with acetosyringone using any of a variety of assays, such as Northern blotting, RT-PCR, real-time amplification, hybridization on microarrays, Western blots, analysis of gene expression from a reporter gene linked to the promoter of a vir gene and the like.

The Ti plasmid may also be transferred to other bacteria without the use of Agrobacterium as a donor strain. For example, a rhizobial strain that has acquired the Ti plasmid by one or another means may act as the donor of the Ti plasmid to other bacterial acceptor strains. This may in some cases avoid the interference of restriction endonuclease systems that exist in many if not all bacteria.

Instead of conjugation, the Ti plasmid may be electroporated into the recipient bacteria. Isolation of the Ti plasmid and electroporation to other Agrobacterium strains, e.g. to the Ti plasmid cured strain LBA288, has been described (Mozo et al., Plant Mol. Biol. 16:617-918, 1990). Similarly, electroporation may be performed to other bacterial species.

For the transfer of the small plasmid or mobilizable binary vector, which is generally a small plasmid, electroporation is conveniently used. The binary vector should be compatible with the Ti plasmid, and both are selected for. Presence of the binary vector may be confirmed by amplification or by re-isolating the plasmid from the bacteria and analysis of the plasmid DNA by restriction digestion.

Transformation of Eukaryotic Cells

Eukaryotic cells may be transformed within the context of this invention. Moreover, either individual cells or aggregations of cells, such as organs or tissues or parts of organs or tissues may be used. Generally, the cells or tissues to be transformed are cultured before transformation, or cells or tissues may be transformed in situ. In some embodiments, the cells or tissues are cultured in the presence of additives to render them more susceptible to transformation. In other embodiments, the cells or tissues are excised from an organism and transformed without prior culturing.

Suitable eukaryotic organisms as sources for cells or tissues to be transformed include plants, fingi, and yeast. Yeast cells can be transformed with Agrobacterium and so can be used in the context of this invention to measure efficiency of transformation and for optimization of conditions. The advantage of using yeast is the fast growth of yeast and the ability to grow it in laboratory conditions. Transformants can be easily detected by their changed phenotype, e.g. growth on a medium lacking an essential growth component on which the untransformed cells cannot grow. Quantization of transformation efficiency is then achieved by counting the number of colonies growing on this selective medium. Yeast cell transformation by Agrobacterium occurs independent of the expression of attachment genes necessary for plant transformation, and, by the use of autonomously replicating DNA units (mini-chromosomes), can avoid the need for gene integration if desired. The uncoupling of attachment and DNA integration from the overall gene transfer processes may simplify the optimization of transformation by other bacteria. For example, following Ti/T-DNA plasmid transfer to these bacteria, the system may be optimized by genetic complementation using an A. tumefaciens genomic library transferred to the pTi-bearing bacteria. The bacterial library is then used to transform yeast cells and the bacterial clones that transform most efficiently are selected.

Alternatively, as Agrobacterium tumefaciens and some of the bacterial species have been fully sequenced and can be compared, missing genes in the latter bacteria that are important for transformation by Agrobacterium may be individually picked from the Agrobacterium genome and inserted into the bacterial genome by any means or expressed on a plasmid. Similarly, the bacteria can be used to transform yeast cells under a variety of test conditions, such as temperature, pH, nutrient additives and the like. The best conditions can be quickly determined and then tested in transformation of plant cells or other eukaryotic cells.

Briefly, in an exemplary transformation protocol, plant cells are transformed by co-cultivation of a culture of bacteria containing the Ti plasmid and the binary vector with leaf disks, protoplasts, meristematic tissue, or calli to generate transformed plants (Bevan, Nucl. Acids. Res. 12:8711, 1984; U.S. Pat. No. 5,591,616). After co-cultivation for a few days, bacteria are removed, for example by washing and treatment with antibiotics, and plant cells are transferred to post-cultivation medium plates generally containing an antibiotic to inhibit or kill bacterial growth (e.g., cefotaxime) and optionally a selective agent, such as described in U.S. Pat. No. 5,994,629. Plant cells are further incubated for several days. The expression of the transgene may be tested for at this time. After further incubation for several weeks in selecting medium, calli or plant cells are transferred to regeneration medium and placed in the light. Shoots are transferred to rooting medium and resulting plants are transferred into the glass house.

Alternative methods of plant cell transformation include dipping whole flowers into a suspension of bacteria, growing the plants further into seed formation, harvesting the seeds and germinating them in the presence of a selection agent that allows the growth of the transformed seedlings only. Alternatively, germinated seeds may be treated with a herbicide that only the transformed plants tolerate.

It is important to note that the bacterial species that are used in this invention may naturally interact in specific ways with a number of plants. These bacterial-plant interactions are very different from the way Agrobacterium naturally interacts with plants. Thus, the tissues and cells that have are transformable by Agrobacterium may be different in the case of the employment of other bacteria. Some plant cell types that are especially desirable to transform include meristem, pollen and pollen tubes, seed embryos, flowers, ovules, and leaves. Plants that are especially desirable to transform include corn, rice, wheat, soybean, alfalfa and other leguminous plants, potato, tomato, and so on.

Uses of Transformation System

The biological transformation system described here can be used to introduce one or more DNA sequences of interest (transgene) into eukaryotic cells and especially into plant cells. The sequence of interest, although often a gene sequence, can actually be any nucleic acid sequence whether or not it produces a protein, an RNA, an antisense molecule or regulatory sequence or the like. Transgenes for introduction into plants may encode proteins that affect fertility, including male sterility, female fecundity, and apomixis; plant protection genes, including proteins that confer resistance to diseases, bacteria, fungus, nematodes, herbicides, viruses and insects; genes and proteins that affect developmental processes or confer new phenotypes, such as genes that control meristem development, timing of flowering, cell division or senescence (e.g., telomerase), toxicity (e.g., diphtheria toxin, saporin), affect membrane permeability (e.g., glucuronide permease (U.S. Pat. No. 5,432,081)), transcriptional activators or repressors, alter nutritional quality, produce vaccines, and the like. Insect and disease resistance genes are well known. Some of these genes are present in the genome of plants and have been genetically identified. Others of these genes have been found in bacteria and are used to confer resistance. Particularly well known insect resistance genes are the genes encoding the crystal proteins of Bacillus thuringiensis. The crystal proteins are active against various insects, such as lepidopterans, Diptera, Hemiptera and Coleoptera. Many of these genes have been cloned. For examples, see, GenBank; U.S. Pat. Nos. 5,317,096; 5,254,799; 5,460,963; 5,308,760, 5,466,597, 5,2187,091, 5,382,429, 5,164,180, 5,206,166, 5,407,825, 4,918,066. Other resistance genes to Sclerotinia, cyst nematodes, tobacco mosaic virus, flax and crown rust, rice blast, powdery mildew, verticillum wilt, potato beetle, aphids, as well as other infections, are useful within the context of this invention. Nucleotide sequences for other transgenes, such as controlling male fertility, are found in U.S. Pat. No. 5,478,369, references therein, and Mariani et al., Nature 347:737, 1990.

Other transgenes that are useful for transforming plants include sequences to make edible vaccines (e.g. U.S. Pat. No. 6,136,320, U.S. Pat. No. 6,395,964) for humans or animals, alter fatty acid content, change amino acid composition of food crops (e.g. U.S. Pat. No. 6,664,445), introduce enzymes in pathways to synthesize vitamins such as vitamin A and vitamin E, increase iron concentration, control fruit ripening, reduce allergenic properties of e.g., wheat and nuts, absorb and store toxic and hazardous substances to assist in cleanup of contaminated soils, alter fiber content of woods, increase salt tolerance and drought resistance, amongst others.

The product of the DNA sequence of interest may be produced constitutively, after induction, in selective tissues or at certain stages of development. Regulatory elements to effect such expression are well known in the art. Many examples of regulatory elements may be found in the Cambia IP Resource document “Promoters used to regulate gene expression” version 1.0, October 2003.

The following examples are offered by way of illustration, and not by way of limitation.

### EXAMPLES

### Example 1

### Identification of Bacterial Species that can Transfer DNA

Divergent bacteria are tested to identify species that are capable of transferring DNA. Strains are obtained from public germplasm banks or isolated from soil, from other natural environments or from any plant tissue. The species is identified by amplification and sequencing of informative genes, including rDNA genes atpD, and recA (Gaunt et al., IJSEM 51:2037-2048, 2001). The DNA sequence of the amplified product is compared to known sequences of specific bacteria. At times, the presence of an amplified product with a predicted size can be used for identification.

As discussed above, suitable bacterial species naturally interact with plants in one or another way. These include endophytic bacteria that live in association with plants, such as rhizobia, which are known to fix nitrogen and make it available to plants. Also included are bacteria that could attach to plants, i.e. epiphytic bacteria, and which have beneficial or neutral interactions with them.

The following bacterial species are tested: Rhizobium spp. NGR234 (a streptomycin-resistant strain ANU240), Sinorhizobium meliloti strain 1021, Mesorhizobium loti MAFF303099, Phyllobacterium myrsinacearum, and Bradyrhizobium japonicum USDA110. All strains are obtained from a public germplasm bank, except for the P. myrsinacearum strain, which is a spontaneous lab isolate.

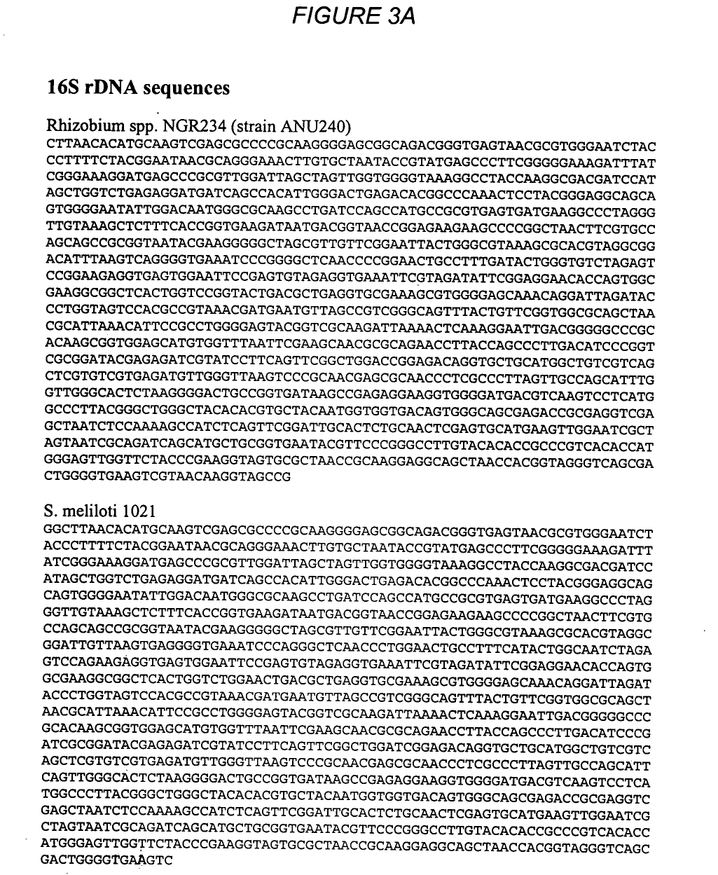

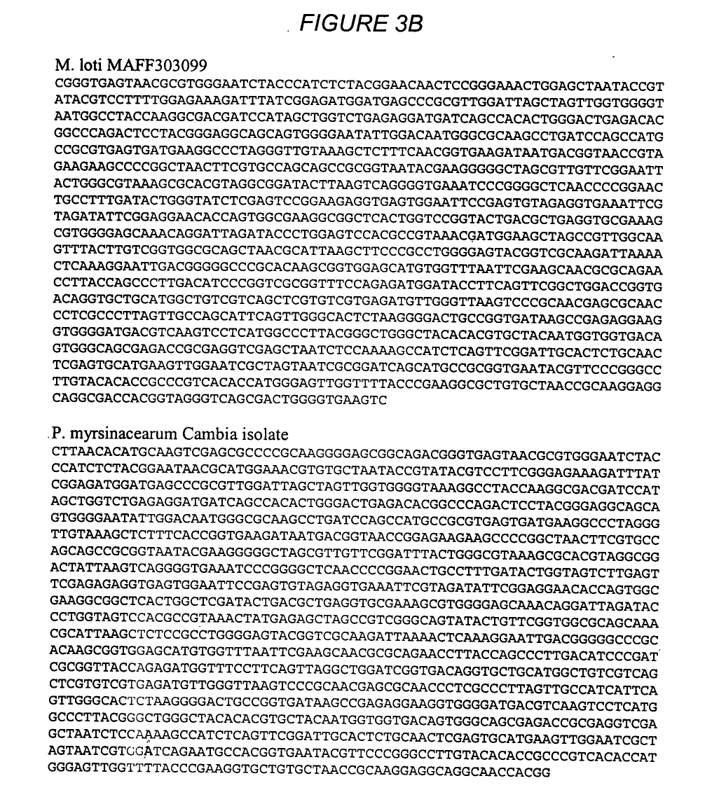

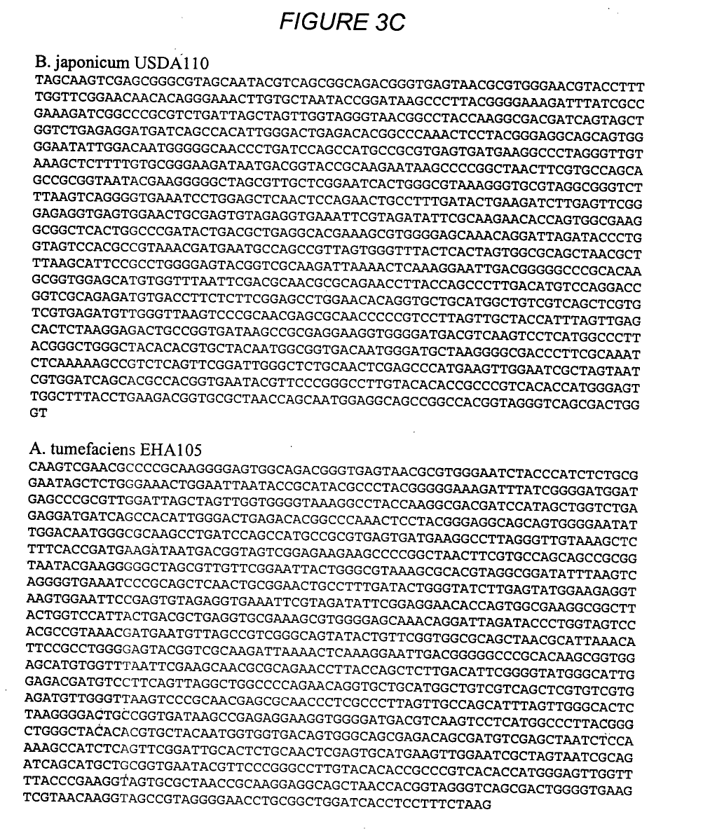

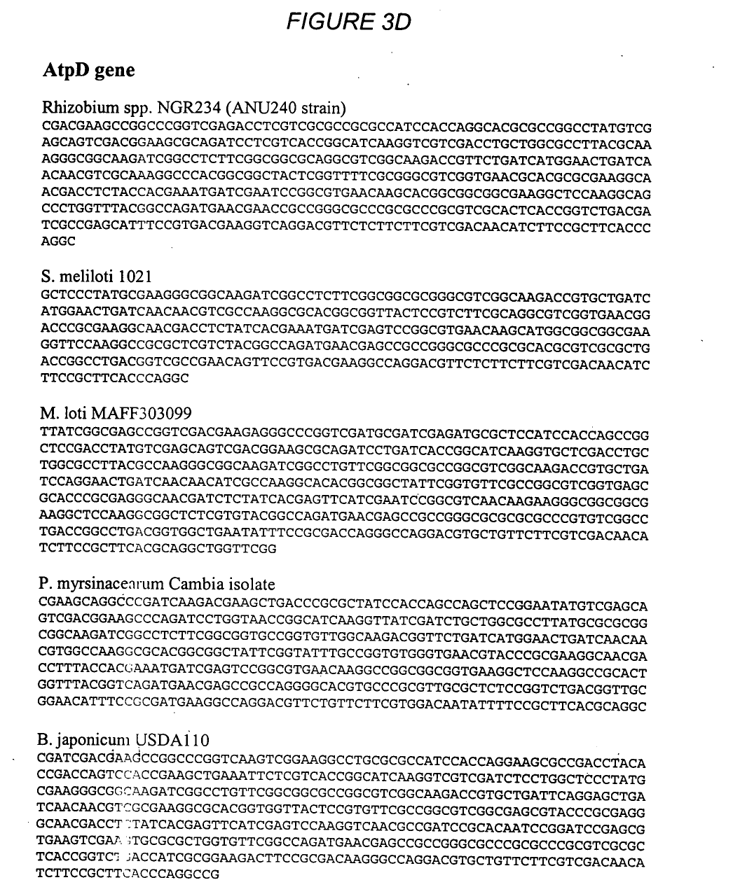

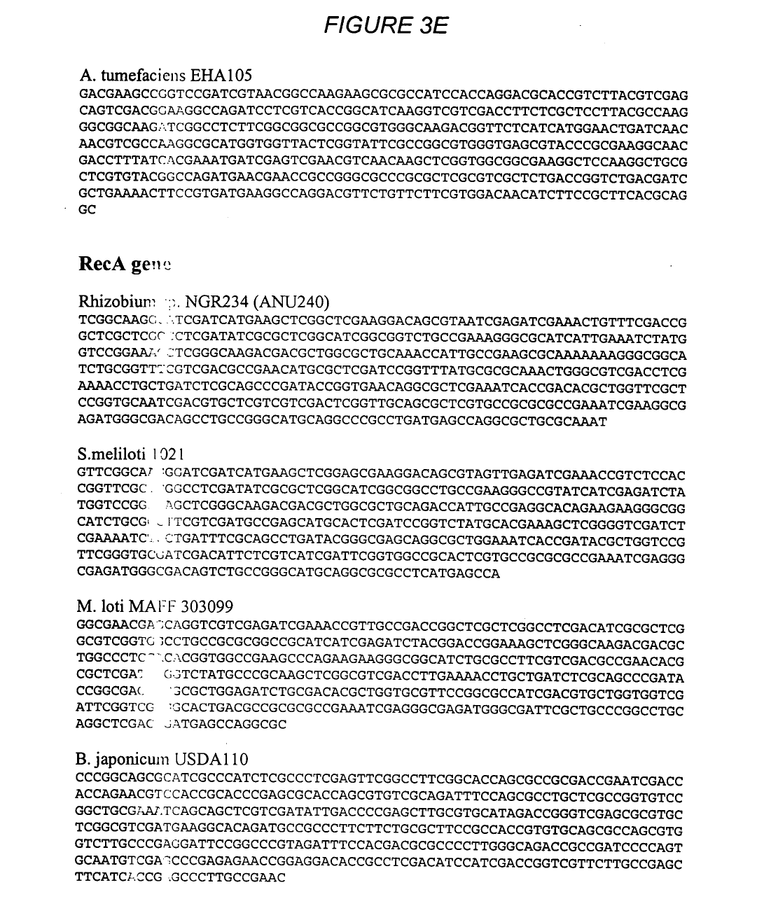

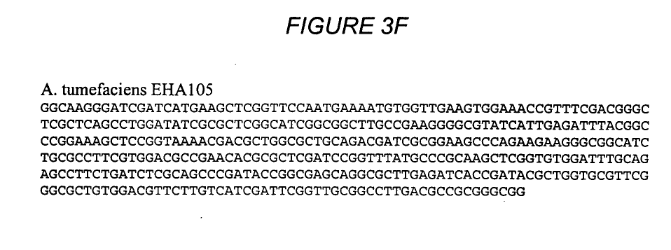

The bacterial species are identified by amplification and sequencing of the 16S rDNA genes and the atpD and recA genes, encoding the beta subunit of the membrane ATP synthase and part of the DNA recombination and repair system respectively (Gaunt et al., IJSEM 51:2037-2048, 2001). The primer sequences that are used to amplify and sequence the partial 16S rDNA genes are SEQ ID NOS:47-50, those for the atpD gene are SEQ ID NOS:51-52, and those for the recA gene are SEQ ID NOS:53-54. The nucleotide sequences that are obtained from sequencing the amplified products generated for the strains assayed are shown in FIG. 3. These sequences, when compared to a database of gene sequences, e.g. GenBank, reveal the highest similarities to Rhizobium spp. NGR234, S. meliloti strain 1021, M. loti MAFF303099, P. myrsinacearum, and B. japonicum USDA110, respectively.

Additional strain identification is done by amplification of informative gene targets on the chromosomal and on the megaplasmid part of the genome and scoring of the presence or absence of the expected amplification product by gel electrophoresis. Such amplification can rapidly confirm the strain genotype during procedures and confirm gain, loss or maintenance of plasmids, such as one or more megaplasmids, often called symbiotic plasmids (pSym) in rhizobia, or a Ti plasmid and a megaplasmid, called the pAT plasmid, in Agrobacterium.

The genotyping primers used here consist of strain- or species-specific primers that amplify at least part of the chromosomally-encoded 16S rDNA genes and other bacterial genes. To design suitable primer sequences, the nucleotide sequences for the targeted gene are retrieved from GenBank and are aligned. Preferably, the aligned sequences include genes from as many bacterial species as possible, and also include those of Agrobacterium tumefaciens. From the alignment, primer sequences are chosen that specifically amplify a sequence from only one or a subset of bacterial species. The species-specific primer pairs are chosen such that the amplified products have a distinct size when separated by gel electrophoresis, allowing their easy scoring during simplex or multiplex reactions.

Chromosomal genes targeted for rapid genotyping include, but are not limited to, the 16S rDNA genes and the attS gene of Agrobacterium tumefaciens, which is present on the circular chromosome. Specific primers for identification of the megaplasmid(s) present in the bacteria include those targeting the NodDI gene on the single pSym plasmid in Rhizobium spp. NGR234, the NodQ and NodQ2 genes present on the pSymA and pSymB plasmids, respectively, of S. meliloti, and the two repA loci present on both M loti megaplasmids, pMLa and pMLb. All of these plasmid primers are designed in such a way that they selectively amplify and hence identify only a particular megaplasmid. Other primers used amplify part of the virG and virB genes on the Ti plasmid of Agrobacterium, and the attS gene copy present on the pAT megaplasmid that is found in most if not all Agrobacterium strains. All primers are chosen to produce an amplification product of a distinct size, allowing easy evaluation of the PCR products on a gel. The primer sequences that are chosen from the alignments of related genes from different bacteria are shown in Table 1.

The templates used for amplification are boiled colonies, obtained by picking some cells from a colony on a plate with a pipet tip, resuspending these into a tube with 100 μL of sterile water, boiling for 3 min and cooling down the crude DNA preparation at room temperature. Then 4 μL of this preparation is used in a 20 μL amplification reaction. Alternatively, purified or more highly enriched DNA can be isolated by any of known methods. All of the primers are rigorously tested on different bacterial species and strains and are employed using the same amplification program, which consists of an initial denaturation of 1 min at 94° C., then 35 cycles of 30 sec at 94° C., 30 sec at 58° C. and 1 min at 72° C., and a final extension for 2 min at 72° C. The products of the amplification reactions are separated by agarose gel electrophoresis, and their sizes are determined by comparison to a ladder of DNA bands of known sizes. The strain assayed is confirmed if the sizes of the products obtained conform to the expected sizes for that strain.

Generally, the bacterial strains are grown on selective media. To find suitable selective growth conditions for the strains tested in this Example, a cell suspension is plated out onto a Yeast Mannitol (YM) agar medium containing one of several different antibiotics (at 25, 50, 100 and/or 200 μg/mL) or rifampicin (100 μg/mL) and incubated for up to 7 days. At least 10⁴ cells are spread per plate. Following incubation, the number of colonies is noted (if <10) or an estimate of the relative growth of the bacteria (+) is scored.

B. japonicum USDA110 is resistant to Gentamycin 25 (25 μg/mL), Rifampicin 100 and moderately to Streptomycin 200. M. loti MAFF303099 is sensitive to all antibiotics tested. S. meliloti 1021 and Rhizobium sp. NGR234 (strain ANU240) are resistant to Streptomycin 200 and slightly to Gentamycin 25 and Rifampicin 100. The P. myrsinacearum strain is resistant to Kanamycin 50, Ampicillin 100, Chloramphenicol 100 and Streptomycin 200. The bacterial strains are also tested for growth on LB agar plates. All bacteria tested, except Rhizobium spp., can grow to a certain extent on an LB plate. Similarly, other media, e.g. synthetic minimal media, can be tested and other antibiotics or growth media components such as different sugars or vitamins can be examined. Preferentially, and to avoid culturing any contaminating microbes, the bacteria are grown under conditions that are selective for the particular strain used. Hence, Rhizobium spp. and S. meliloti are grown on YM+strep200, P. myrsinacearum on YM+Km50, B. japonicum on YM+Rif100 and M. loti on plain YM plates.

In order to find suitable conditions for the elimination of bacteria following a plant transformation experiment, the bacterial strains are grown on plates containing different concentrations of cefotaxim, timentin and moxalactam, three commonly employed antibiotics to counterselect against Agrobacterium. The results show complete inhibition of growth of all strains tested, except S. meliloti, with low concentrations of cefotaxime (50 μg/mL); growth of S. meliloti can be inhibited with Moxalactam at 200 μg/mL or with a combination of cefotaxime and timentin (both at 100 μg/mL).

TABLE 1

| | GENOMIC | | | | LENGTH | PRODUCT | SEQ ID |
|---|---|---|---|---|---|---|---|
| SPECIES/STRAIN | LOCATION | GENE | PRIMERS | SEQUENCE 5′-3′ | (nts) | SIZE (BP) | No. |
| A. tumefaciens | Chromosome | 16S rRNA | Atu16SFW2 | | 23 | 320 | 22 |
| | (circ. + | | Atu16REV | CGGGGCTTCTTCTCCGACT | 19 | | 21 |
| | linear) | | | | | | |
| | Circular | AttS | attScircFW | CAGGCTCAAACCGCATTTCC | 20 | 436 | 23 |
| | chromosome | | attScircREV | GTAAGTCCAGCCTCTTTCTCA | 21 | 24 | |
| | Ti plasmid | VirG | AtuvirGFW | CGCTAAGCCGTTTAGTACGA | 20 | 520 | 27 |
| | | | AtuvirGREV | CCCCTCACCAAATATTGAGTGTAG | 24 | | 28 |
| | | downstream of | VirBFW | TGACCTTGGCCAGGGAATTG | 20 | 947 | 31 |
| | | virB operon | VirBREV | TCCTGTCATTGGCGTCAGTT | 20 | | 32 |
| | | NptI (only in | NptIFW | CAGGTGCGACAATCTATCGA | 20 | 633 | 29 |
| | | EHA101) | NptIREV | AGCCGTTTCTGTAATGAAGG | 20 | | 30 |
| | AT plasmid | AttS | attSpATFW | GTGCTTCGGATCGACGAAAC | 20 | 631 | 25 |
| | | | attSpATREV | GGAGAATGGGAGTGACCTGA | 20 | | 26 |
| Rhizobium sp. | Symbiotic | NodD1 | NGRNodD1FW | GCCAGAAATGTTCATGTCGCACA | 23 | 350 | 35 |
| NGR234 (ANU24O) | plasmid | | NGRNodD1REV | AATGGGTTGCGGAAGTTCGGT | 21 | | 36 |
| S. meliloti1021 | Chromosome | 16S rRNA (1) | Sme16SFW | TGTGCTAATACCGTATGAGC | 20 | 820 | 33 |
| | | | Sme16SREV | CAGCCGAACTGAAGGATACG | 20 | | 34 |
| | pSymA | NodQ | SmeNodQFW | GACAGGATCCTCCACGCTCA | 20 | 420 | 37 |
| | | | SmeNodQREV | CGCCAGGTCGTTCGGTTGG | 18 | | 38 |
| | pSymB | NodQ2 | SmeNodQFW | GACAGGATCCTCCACGCTCA | 20 | 360 | 37 |
| | | | SmeNodQ2REV | GCTCATAGGGCGAGGATACA | 20 | | 39 |
| M loti | Chromosome | 16S rRNA | Mlo16SW | CCCATCTCTACGGAACAACT | 20 | 500 | 55 |
| MAFF303099 | | | Mlo16SREV | ACTCACCTCTTCCGGACTCG | 20 | | 56 |
| | pMLa | RepC | MlopMLaRepCFW | GACGGCCGAGCCAAGGACGA | 20 | 200 | 57 |
| | | | MlopMLRepCREV | CACATGGCAAGCCTCCTCA | 19 | | 58 |
| | pMLb | RepC | MlopMLbRepCFW | GATGCTGGAAAGCTTCACAAGT | 22 | 320 | 59 |
| | | | MlopMLRepCREV | CACATGGCAAGCCTCCTCA | 19 | 58 | |
| P. myrsinacearum | Chromosome | 16SrRNA | Pmy16SFW | CTGGTAGTCTTGAGTTCGAG | 20 | 400 | 60 |
| strain WB1 | | | Pmy16SREV | CCAGGCTAACTGAAGGAAAC | 20 | | 61 |
| | | DNA Gyrase | PmyGyrBFW | CTGGCTGCGTCTCAAGATTC | 20 | 544 | 62 |
| | | B | PmyGyrBREV | CCTTTGCCTTCTTCGCCTGC | 20 | | 63 |
| B.japonicum | Chromosome | 16S rRNA | Bja16SFW | GGGCGTAGCAATACGTCA | 18 | 600 | 64 |
| USDA110 | | | Bja16SREV | CTTCGCCACTGGTGTTCTTG | 20 | 65 | |

(1) these primers also amplify the 16S rRNA gene in the NGR234 strain ANU240

### Example 2

### Identification of Agrobacterium Strains that can Serve as Donor of the Ti Plasmid, Isolation of the Ti Plasmid and Transfer to Other Bacteria by Electroporation

The Agrobacterium strain that is used as a source of the Ti plasmid is the hypervirulent strain EHA105, which contains the Ti plasmid pEHA105, a disarmed derivative of pTiBo542 (Hood et al., Transgenic Research 2:208-218, 1993). To confirm the strain, Agrobacterium-specific genotyping primers are designed for the 16S rDNA genes (SEQ ID NOS:22-23) and for the attS genes on either the circular chromosome (SEQ ID NOS:23-24) or on the pAT megaplasmid (SEQ ID NOS:25-26). Primers are also designed to amplify sequences on the Ti plasmid, i.e. for the virG (SEQ ID NOS:27-28) and virB genes (SEQ ID NOS:31-32). These primers are tested for the specific and efficient amplification of Agrobacterium DNA. They are also tested on DNA templates prepared from all the other bacterial species that are assayed for gene transfer. The results show specific amplification of Agrobacterium DNA, but no detectable amplification from other bacterial templates.

The same primer sets can be used to confirm absence of Agrobacterium cells in bacterial cultures, suspensions or any other preparations used during plant transformation. To determine the minimum number of Agrobacterium cells detectable in a culture of another bacterial species, the following experiment is done. A culture of Rhizobium leguminosarum biovar trifolii (strain ANU843), a close relative of Agrobacterium, is grown to an OD600 of 1.0, corresponding to 10⁸-10⁹ cells/mL, in TY (Tryptone-Yeast Extract) medium at 29° C. A culture of A. tumefaciens EHA101 is grown in LB medium with Km50 at 29° C. and diluted in 10-fold steps. The number of cells in each of the dilutions is determined by plating an aliquot onto LB agar plates and counting the number of cells. From these calculations, the number of cells per mL is determined and serial dilutions containing 20, 200, 2000 and 20.000 cells in a volume of 10 μL are prepared. Then 4 tubes are prepared containing 10 μL of the 10-fold diluted rhizobial culture, corresponding to 2×10⁵ cells, and 80 μL of sterile water; then, 10 μL from each of the Agrobacterium dilutions is added, such that each tube contains 2, 20, 200 and 2000 Agrobacterium cells respectively. A fifth tube is made by addition of 2000 Agrobacterium cells in a total volume of 100 μL of water, without Rhizobium cells. All tubes are held in a boiling water bath for 3 min to lyse the cells and release the DNA.

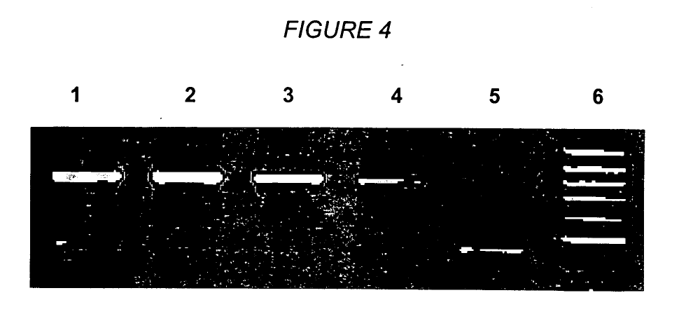

Amplification is performed using 10 μL of template DNA from tube 1 to 5 in a total volume of 20 μl. The amplification mixtures contain two sets of primers (duplex amplification), one specific for the R. leguminosarum 16S rDNA genes (SEQ ID NOS:18-19) and one specific for the A. tumefaciens 16S rDNA genes (SEQ ID NOS:20-21), which amplify the partial 16S rDNA genes in R. leguminosarum and A. tumefaciens respectively and yield products of a different size upon gel electrophoresis (approx. 700 and 410 bp respectively). The amplification reactions are carried out using an initial denaturation temperature at 94 C during 1 min, then 40 cycles of 30 sec at 94 C, 30 sec at 58 C, 1 min at 72 C, and a final extension at 72 C during 2 min. The reaction products are separated by electrophoresis and visualized by ethidium bromide staining. The results are shown in FIG. 4. Amplified Rle16S sequence (700 bp) is detectable in all samples containing Rhizobium DNA. The Atu16S band (410 bp) is seen in the control sample 5 (lane 5), and in samples 1 to 3 with decreasing intensity (lanes 1 to 3), but not in lane 4. The limit of detection of Agrobacterium in a non-Agrobacterium culture thus corresponds to 2 Agrobacterium cells in a 20 μL amplification reaction

To isolate the Ti plasmid for electroporation to other bacteria, a 2 mL culture of EHA101 is grown to an OD600 of 1.0 in LB+Kanamycin 50 μg/mL. EHA101 is very similar to EHA105, but contains the NptI gene which confers kanamycin resistance to this strain (Hood et al., J. Bacteriol. 168:1291-1301, 1986). Plasmid DNA is isolated by a modified alkaline lysis method that is adapted for isolation of large plasmids. The culture is diluted 20× into fresh medium and grown for another 2 to 3 h. The cells are harvested by centrifugation (2500×g, 10 min) and resuspended in 2 mL of TE (10 mM Tris, pH 8 and 1 mM EDTA) buffer, pelleted again and resuspended in 40 μL of TE. Freshly prepared lysis buffer (4% SDS in TE pH 12.4), 0.6 mL, is added to a 1.5 mL Eppendorf tube and the bacterial cells are pipetted into this lysis solution and carefully mixed. The suspension is incubated for 20 min at 37° C., then neutralized by adding 30 μL of 2.0M Tris-HCl pH 7.0 and slowly inverting the tube until a change in viscosity is noted. The chromosomal DNA is then precipitated by adding 240 μL of 5M NaCl and incubating the tubes on ice for 1 to 4 hr. After centrifugation for 10 min at 16000×g, the supernatant is poured into a new tube, and 550 μL of isopropanol is added to precipitate the plasmid DNA. The tube is placed at −20° C. for 30 min, then centrifuged at 16000×g for 3 min. The supernatant is removed, and the pellet dried at room temperature. The pellet is resuspended in 10 μL TE by overnight incubation at 4° C.

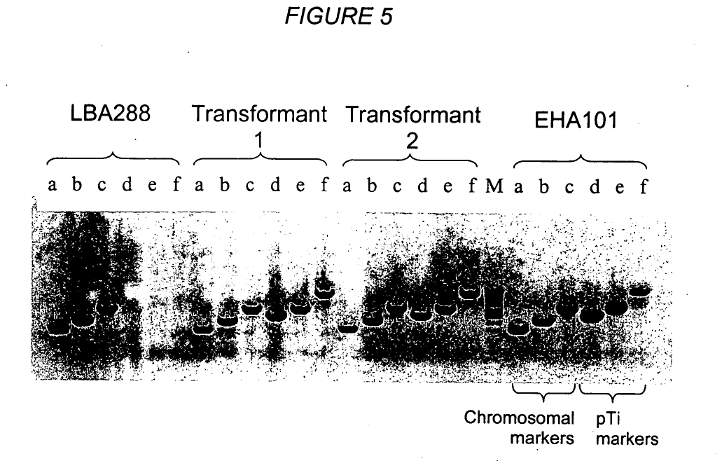

The Ti plasmid is transferred to other bacteria by electroporation. Here we show pTi transfer to the Agrobacterium strain, LBA288, which is cured for the Ti plasmid. Electrocompetent cells are prepared from exponentially grown cells according to standard procedures for A. tumefaciens. 40 μl of thawed competent cells are added to the tube containing 10 μl of resuspended EHA101 plasmid DNA, slowly mixed, and transferred to an chilled microcuvette (Bio-Rad, 0.1 cm electrode distance). A single electric pulse of 5 ms at a field strength of 13 kV/cm is applied by means of the Gene Pulser and Pulse Controller of Bio-Rad. Due to their large size, lower field strengths are generally used during electroporation to increase the efficiency for transfer of Ti plasmids. Immediately following the electric pulse, 600 μl of SOC is added and the cell suspension is transferred to an 1.5 mL Eppendorf tube and incubated for 1 hr. Then 100 μL aliquots are spread onto LB agar plates containing Rifampicin 50 (for LBA288) and Kanamycin 50 (for the Ti plasmid). After 2 days incubation at 28 C, colonies are observed on the plates. Amplification is carried out on a number of colonies to examine the presence of the Ti plasmid from EHA101. FIG. 5 shows the results of the analysis on two independent transformants and the donor and acceptor strain using primers for the chromosomes, the pAT plasmid and the Ti plasmid. The results reveal that the LBA288 strain has acquired the Ti plasmid of EHA101. Likewise, the Ti plasmid can be electroporated to other bacterial species using the specific electroporation conditions suitable for every species. Functionality of the Ti plasmid is shown by plant transformation experiments.

### Example 3

### Construction of a Mobilizable Ti Plasmid

Although the Ti plasmids are generally self-conjugative plasmids, their mobilization under laboratory conditions is cumbersome due to the absence of the specific components and conditions necessary to activate their conjugation machinery. In this example, the disarmed Ti plasmid from EHA105 is made transmissible by insertion of the origin of transfer (oriT) of the RP4/RK2 helper plasmid. As well, an antibiotic resistance marker is inserted in the Ti plasmid in order to be able to select for transconjugants. The resulting modified Ti plasmid can then be mobilized through the transfer functions provided by the RP4/RK2 plasmid and selected for.

The RP4 oriT is inserted into a Ti plasmid utilizing a vector that inserts into the Ti plasmid by homologous recombination. Several types of vectors can be used, such as suicide vectors or broad host range vectors. Suicide vectors contain an origin of replication that is not functional in Agrobacterium and one or more antibiotic selection markers. Selection for these markers forces the suicide vector to recombine into the genome, e.g. into the Ti plasmid. Other suitable vectors contain a broad host-range origin of replication that is stable in Agrobacterium (e.g. RK2). The latter is forced to insert into the Ti plasmid by transformation of the strain with a plasmid that is incompatible with the broad host-range vector and selection for both plasmids. Homologous recombination is enhanced by cloning a region of the Ti plasmid into the suicide or broad host-range vector, thereby allowing this region to recombine with the same sequence on the Ti plasmid.

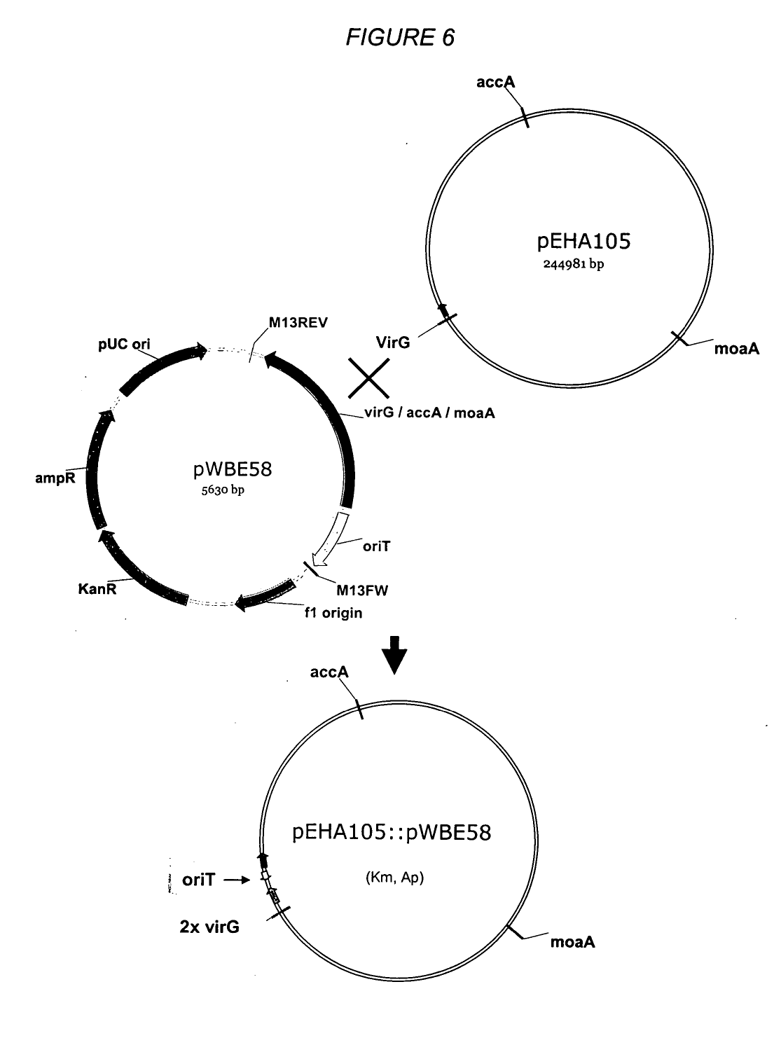

In this example a suicide vector is used that is derived from the Topo vector PCR2.1 (Invitrogen, Carlsbad, Calif.). A sequence of the Ti plasmid that will function as a target for homologous recombination is amplified and T/A cloned into this Topo vector. The target sequence encompasses the whole virG gene flanked by partial sequences from the virB11 and virC2 genes respectively (primer sequences VirB11FW and VirC2REV; SEQ ID NOS:66-67)). Two other suicide vectors are constructed by T/A cloning of partial sequences from the moaA gene, using primers moaAFW and moaAREV (SEQ ID NOS:68-69), and partial sequences from the accA gene using primers accAFW and accAREV (SEQ ID NOS:70-71), respectively. These three genes are located on different positions along the Ti plasmid sequence and recombination with the suicide vectors will thus result in modifications to the Ti plasmid in three different regions (in separate Ti plasmids). The resulting suicide vector constructs are confirmed by sequencing. Then the RP4 oriT sequence is amplified from plasmid pSUP202, a derivative of the RP4 vector, using primers oriTFW and oriTREV (SEQ ID NOS:72-73). The oriT product is cloned into the Xba I site of the three suicide vectors, transformed to E. coli Top10 competent cells and the plasmid vectors are confirmed by sequencing. The vector maps for one of the suicide plasmids, pWBE58, is shown in FIG. 6 along with the strategy used for homologous recombination into the Ti plasmid of EHA105. The suicide vectors are then electroporated to Agrobacterium tumefaciens EHA105. Putative transformants with vector integrants are selected on LB plates supplemented with Km50 and Cb100 (both selection markers are present on the suicide vectors). Candidate colonies that have integrated the suicide vector into the Ti plasmid by homologous recombination at the virG, accA or moaA locus are obtained in 3 days and assayed by amplification for the presence of the modified Ti plasmid.

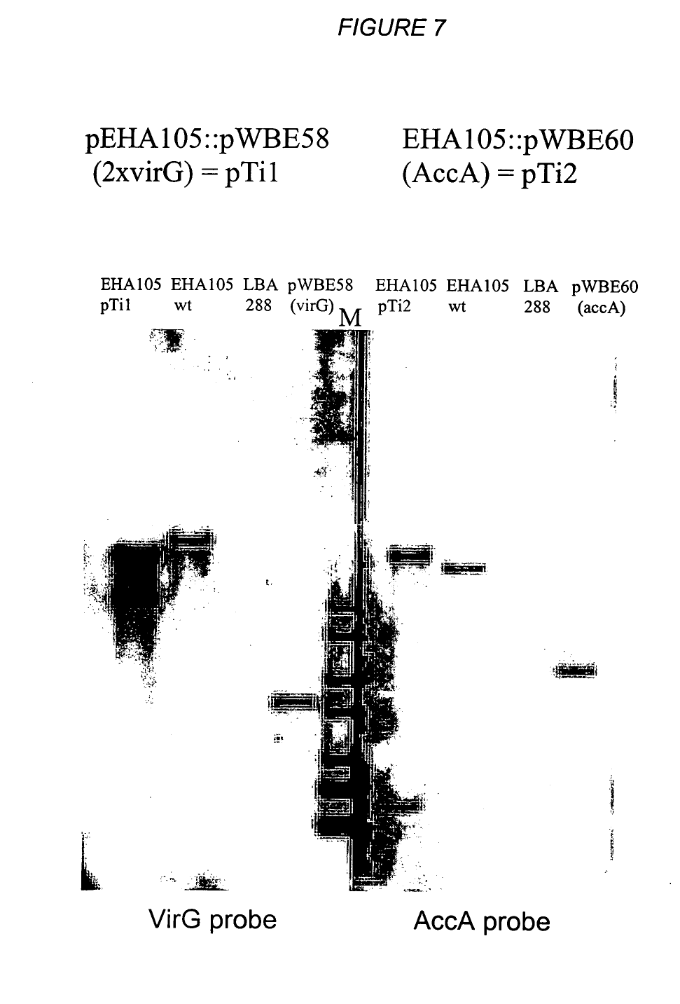

Primers used to verify integration of the whole suicide plasmid into the Ti plasmid are as follows: virB11FW2 (SEQ ID NO:40) and M13REV (SEQ ID NO:41) for the pTi::pWBE58 integrant, now called pTi1, accAFW2 (SEQ ID NO:74) and M13REV (SEQ ID NO:41) for the pTi::pWBE60 integrant, now called pTi2, and M13FW (SEQ ID NO:42) and moaAREV2 (SEQ ID NO:75) for the pTi::pWBE62 integrant, now called pTi3. In each case, the M13 primer anneals to the suicide vector sequence and the second primer anneals to a sequence outside the region cloned in the respective suicide vectors. Amplification is carried out using an initial denaturation at 94 C for 1 min, then 35 cycles of 30 sec at 94 C, 30 sec at 58 C and 2 min at 72 C, and a final extension for 2 min at 72 C. The amplified products are separated by agarose electrophoresis. The results (FIG. 7) show the presence of the expected amplification products for each of the vector integrations: a 1496 bp product for pTi1, 2080 bp for pTi2, and 1627 bp for pTi3, respectively. No amplification product is obtained for the wildtype EHA105 strain containing an unmodified Ti plasmid.

Further evidence for integration of the suicide vectors in the Ti plasmid is obtained by Southern blot analysis. Genomic DNA is isolated from the wildtype EHA105 strain, from the Ti plasmid-cured Agrobacterium strain LBA288, and from the EHA105 strains containing modified Ti plasmids pTi1 and pTi2. The genomic DNA is digested by the restriction endonuclease XbaI and separated by gel electrophoresis run overnight. XbaI cuts the suicide vectors twice, once at each side of the oriT sequence. In the modified Ti plasmid sequence, this should result in the cleavage of the DNA inside the duplicated virG and accA region respectively, resulting in two fragments each containing a virG or accA fragment. The digested genomic DNA is then blotted onto a membrane, fixed and hybridized to a DNA probe. In a separate lane, the XbaI-digested suicide vector DNA is loaded. The DNA probe is prepared by DIG labeling (HighPrime DIG labeling kit, Roche diagnostics, Mannheim, Germany) of an amplified product corresponding to the virg gene and the accA gene amplified from the corresponding suicide vectors by using the M13 primers (SEQ ID NOS:41-42) and the accAFW+accAREV primers (SEQ ID NOS:70-71) respectively. Development of the film following exposure to the hybridized and washed membrane reveals the presence of a single band in the wildtype strain, and two bands in the pTi1 and pTi2 strains. The LBA288 strain which does not have a Ti plasmid shows no bands for either of the probes, indicating that the probes bind to a region of the Ti plasmid. The result confirms that the whole suicide vectors have integrated into the homologous region of the Ti plasmid by a single cross-over event, thereby duplicating the region that was cloned in the vectors (virG and accA respectively). This is shown in FIG. 7. In pTi1, this results in the duplication of the whole virG gene, while in pTi2, a second truncated copy of the AccA gene is inserted. In Agrobacterium, strains with duplicated virG genes or enhance virG activity have been shown to have increased gene transfer competence.

### Example 4

### Transfer of the Ti Plasmid to E. Coli and Other Bacteria and Manipulation of the Ti Plasmid in E. Coli

In this example, the Ti plasmid is transferred to E. coli cells and maintained and modified in E. coli. (Hille et al., J. Bacteriol. 154:693-701, 1983) showed that a spontaneous stable cointegrate between a wildtype octopine Ti plasmid and the wide-host range plasmid R722 could be maintained in E. coli. The disarmed Ti plasmid EHA105 is modified by insertion of a RK2 origin of replication and origin of transfer and transferred to E. coli by electroporation or conjugation.

The unmodified Ti plasmid is unstable in some bacterial species. Thus, in one embodiment of this invention, the Ti plasmid is modified by insertion of a broad-host range origin of replication, thereby making it more stable and replicative in other bacterial species, including but not limited to E. coli. The modified Ti plasmid is then conjugated to non-Agrobacterium species, for example to Bradyrhizobium japonicum or Azospirillum brasilense. Any replication origin or stabilization protein gene that is stably maintained in a species can be employed for stabilizing the Ti plasmid.

The Ti plasmid is first modified by insertion of a replicative origin that is active in E. coli. The broad-host range plasmid pRK404, a smaller derivative of RK2 (Scott et al., Plasmid 50:74-79, 2003; GenBank accession AY204475), was modified by replacing the tetracycline resistance genes (tetA and tetR) by the kanamycin resistance gene from Topo vector PCR2.1 (Invitrogen, Carlsbad, Calif.). pRK404 was digested with BseRI, and the large fragment blunted with T4 DNA polymerase and ligated to the EcoRV/XmnI fragment containing kanR and the F1 ori from PCR2.1. The resulting 10.5 kb vector is kanamycin resistant and is called pRK404 km. To favor homologous recombination with the Ti plasmid, a sequence of the Ti plasmid is cloned into the pRK404km vector. The whole virG gene and part of the moaA gene with flanking DNA are amplified using primers virB11FW and virC2REV (for virG; SEQ ID NOS:66-67)), and primers moaAFW and moaAREV (for moaA; SEQ ID NOS:68-69), all of which carry restriction sites. The amplified products are digested with HindIII (virG) or BamHI (moaA) and ligated to the similarly digested pRK404 km plasmids. Ligation reactions are electroporated into E. coli and transformants growing on kanamycin50 and remaining white in the presence of X-gal and IPTG are analysed for the presence of the expected plasmids. The resulting vectors are then electroporated to wild-type EHA105 competent cells and transformants are selected on kanamycin50. Alternatively, the pRK404 km/virG or pRK404 km/moaA plasmids are conjugated to EHA105 in a triparental mating with the help of RP4-4 provided by another E. coli strain, or in a biparental mating using the E. coli strain S17-1 (which has the RP4 transfer functions integrated in its chromosomes) to which the pRK404 km/virG or pRK404 km/moaA plasmids have been electroporated.

The resulting EHA105 transformants most probably carry the pRK-derived plasmid vectors as a separate plasmid. In order to force these vectors to integrate into the Ti plasmid, the strains are transformed with another incP plasmid, which is incompatible with the former vectors, and transconjugants/integrants are selected for both the kanR gene on the initial pRK vector and the selection marker on the second incP vector.

The EHA transformants are transformed by conjugation with an E. coli strain carrying RP4-4 (derivative of RP4 which is Kan-sensitive) and selected on M9 sucrose (to counterselect against E. coli) plates with Kan50 and Carbenicilin100. Among the resulting transconjugants, some colonies will have the pRK-vector integrated in the virG or moaa sequence regions of the Ti plasmid and additionally carry the RP4-4 vector. These colonies are then used for conjugation experiments to E. coli, in which the E. coli transconjugants are selected on LB plates containing kan50 at 37° C. The resulting E. coli colonies may have acquired the RP4-4 plasmid in addition to the Ti plasmid. A number of colonies are plated several times onto fresh plates and spontaneous loss of the RP4-4 plasmid is checked by replica plating onto LB with Carb100. The presence of the Ti plasmid in these E. coli strains is confirmed by amplification using primers for the Ti plasmid markers virG, virB and moaa (SEQ ID NOS:27-28; 31-32; and 68-69 respectively).

The Ti plasmid in E. coli can be manipulated by any of the commonly used tools for genetic manipulation in Gram-negative bacteria, including transposon mutagenesis and lambda recombinase-supported homologous recombination. Large parts may be deleted from the Ti plasmid in regions that are unnecessary for gene transfer to plants. Sequences may be inserted to increase stability, maintenance or gene transfer ability of the Ti plasmid. The modified Ti plasmid is then transferred back into a suitable bacteria strain by electroporation or conjugation methods and used for transformation of plants or other eukaryotes.

### Example 5

### Construction of “Marked” Binary Vectors for Plant Transformation by A. Tumefaciens and Non-Agrobacterium Bacteria

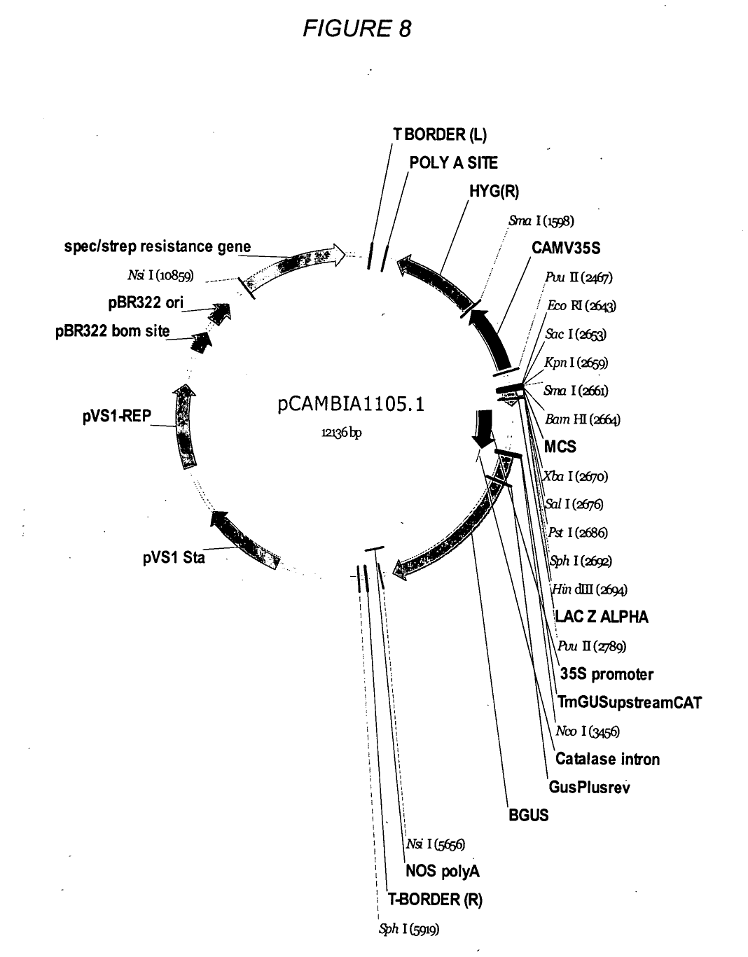

The binary vector system is employed for gene transfer to plants. The bacterial vehicle to transfer a DNA sequence of interest to plants therefore contains a disarmed Ti plasmid without T-DNA and a vector that contains the gene(s) of interest between T-DNA borders. The vector that is used here is derived from the pCAMBIA series of vectors, i.e. from pCAMBIA1305.1 (GenBank Accession: AF354045). The vector is modified by replacement of the kanamycin resistance marker npti by the spectinomycin/streptomycin resistance marker (SpecR) from pPZP200 (Hajdukiewicz et al., Plant Molec. Biol. 25:989-994, 1994). The SpecR gene is amplified from pPZP200 by primers SpecFWNsiI (SEQ ID NO. 76) and SpecREVSacII (SEQ ID NO. 77), digested with NsiI and SacII and ligated to both large fragments from a pCAMBIA1305.1 NsiI/SacII digest, leaving out the 988 bp fragment that contains the KanR gene. The resulting vector, after checking the correct orientation of the ligated fragments, has the SpecR gene replacing the KanR gene and is called pCAMBIA1105.1. A map of this vector is shown in FIG. 8. It contains all the features of pCAMBIA1305.1, including the hygromycin resistance cassette and the GusPlus (U.S. Pat. No. 6,391,547) reporter gene cassette within the left and right T-DNA borders. The GusPlus gene contains an intron, preventing it from being expressed in the bacteria. Following X-gluc staining of a bacterial suspension, no blue spots are detected. Similarly, pCAMBIA1405.1 is constructed by amplification of the Spec gene from pPZP200 with SpecfwSacII and SpecrevSacII (SEQ ID NOS:78+77) and ligation into the unique SacII site of pCAMBIA1305.1. This vector, pCAMBIA1405.1, has a combined Kan and Spec resistance and contains exactly the same T-DNA region as its parental vector and pCAMBIA1105.1.

In order to verify that gene transfer has occurred through the help of the non-Agrobacterium species and not through contaminating Agrobacterium cells, a slightly different binary vector is transformed to the bacteria of this invention compared to the one transformed to Agrobacterium strains that are used as a positive control during transformation. To mark the binary vector and have this marker sequence be integrated into the target plant species' genome, a small part of the T-DNA region is modified, e.g., a slightly different multi-cloning site is used in both vectors or small deletions or insertions are created in any region within the border sequences. One binary vector, here called the “marked binary vector” (MBV), is transformed to the non-Agrobacterium strain only, and will never be introduced into any of the Agrobacterium strains. The other binary vector (BV) is introduced in Agrobacterium strains only. Transformed plant tissues can be analysed for the type of T-DNA sequence that has integrated into the genome by amplification across the marker sequence and determining the DNA sequence of the product. Any T-DNA integration can thus be examined by amplification and preferably by sequencing. Thus, the origin of the T-DNA can be identified as being derived from either the target bacterium strain or from Agrobacterium.

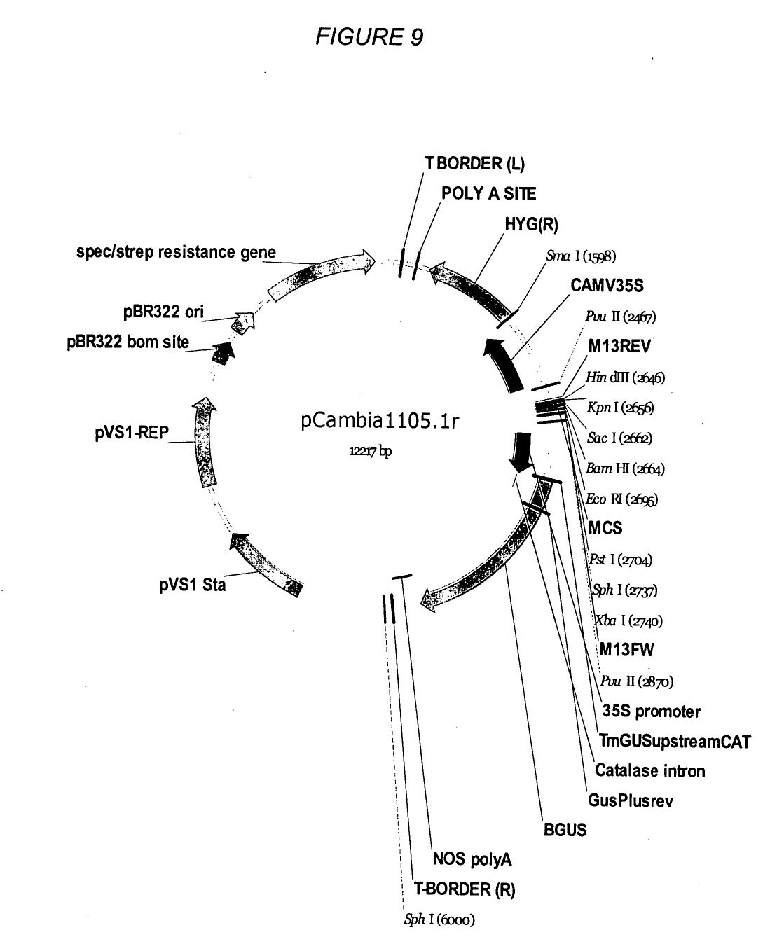

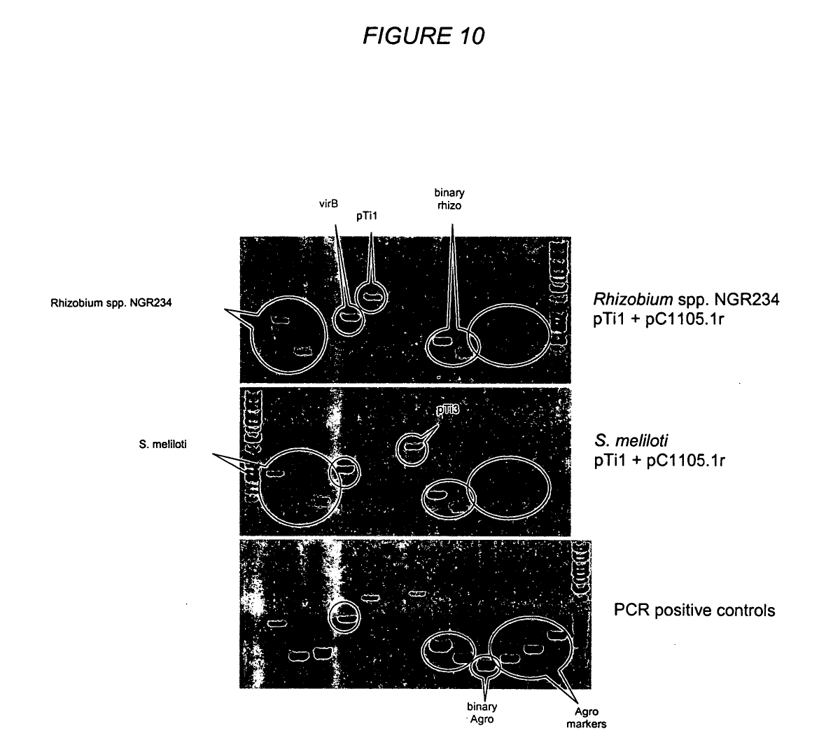

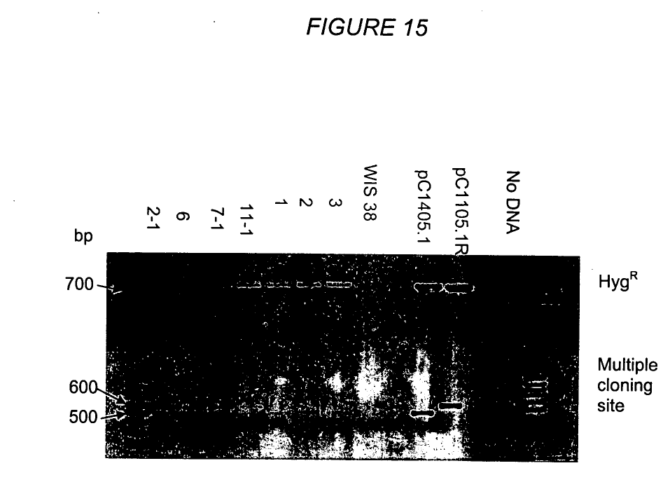

In this example, the pCAMBIA1105.1 vector is marked by replacing its multi-cloning site by the slightly different one from Topo vector PCR2.1 (Invitrogen, Carlsbad, Calif.). The multi-cloning site from the Topo vector is cut out as a PvuII fragment and ligated into PvuII-digested pCAMBIA1105.1. The resulting vector is analysed by amplification across the multi-cloning site sequence and by sequence analysis of the whole multi-cloning site. The marked vector is called pCAMBIA1105.1R (FIG. 9) and is electroporated only to the bacteria of this invention. Similarly, the original vector, pCAMBIA1105.1, or the related vectors pCAMBIA1305.1 and 1405.1, are only electroporated to Agrobacterium, and the resulting strains are used as a positive control for gene transfer. The different MCS sequences in the marked binary vector compared to the original vector is confirmed by amplification of the MCS with primers 1405.1 (SEQ ID NO. 46) and P35S5′rev (SEQ ID NO. 79), yielding a 491 bp product for the 1105.1/1305.1/1405.1 series of vectors and a 572 bp product for the marked binary vector pCAMBIA1105.1R. This is shown in FIG. 10 and FIG. 15.

### Example 6

### Construction of Bacterial Strains that can Transfer DNA

In this example, bacterial strains are engineered for DNA transfer by incorporation of the Agrobacterium Ti plasmid and a T-DNA binary vector. The Ti plasmid is first transferred from Agrobacterium to a bacterial strain of this invention by conjugation. The pTi helper plasmid has strong virulence functions, e.g. pEHA105 from EHA105, and bears a positive selection marker(s). In one embodiment, the mobilization of the Ti plasmid is accomplished by the help of the conjugation machinery of RP4/RK2 plasmids. These IncP plasmids, or derivatives thereof, are able to mobilize a plasmid that carries the origin of transfer (oriT) of RP4/RK2 (see Example 3). If the bacterial strain of this invention strain has no useful selection marker, a selection marker is first inserted in its genome by transposon-mediated mutagenesis or by any recombination approach.

EHA105 carrying pTi1 and EHA105 carrying pTi3 (both pTis carry resistances to kanamycin and carbenicillin; see Example 3) are used as donor strains. E. coli carrying RP4-4 (a kanamycin-sensitive derivative of RP4) or E. coli carrying pRK2073 (a spectinomycin-resistant RP4 derivative containing the RP4 transfer functions on a limited host range replicon that is not active in Agrobacterium or the strains of this invention) are used as a helper strain, Rhizobium spp. NGR234 (streptomycin-resistant strain ANU240) and Sinorhizobium meliloti strain 1021 (streptomycin resistant) are used as acceptor strains.

Conjugation is brought about by combining actively growing cultures of the donor Agrobacterium strain containing the Ti plasmid, the rhizobial acceptor strain and the helper RP4/RK2 (derivative) strain in a triparental mating. Bacterial mixes are transferred to a nitrocellulose filter placed on a nonselective YM growth medium and incubated for few hours or overnight at 29° C. Cells on the filter are then resuspended and plated onto selective plates (YM with Strep100, Kan50 and Cb50) that favor the growth of the transconjugants, that is the rhizobia containing the Ti plasmid. The candidate transconjugants are plated out as single cell colonies and checked by amplification for the presence of the pTi (e.g. vir genes) and confirmed as the rhizobial strain. The results of the amplification analysis for one strain of each bacterial species are shown in FIG. 10. The transconjugant strains are additionally analysed for the presence of the RP4-derived helper plasmid (using primers RP4FW and REV; SEQ ID NOS: 80-81). A strain is chosen for further use that lacks this plasmid.

The rhizobial strains containing the Ti plasmid are then transformed with pCAMBIA1105.1R (see Example 4) by electroporation. The putative transformants are selected on YM media containing Km50 (to select for the pTi) and Sp100 (to select for the binary vector). Candidate colonies are observed after 3-5 days, plated onto new plates and analysed by amplification for the presence of the binary vector (primers for hygR, SEQ ID NOS:44-45, and the multi-cloning site, SEQ ID NOS:46+79), the Ti plasmid (virg, virB and moaA primers, SEQ ID NOS:27-28; 31-32; 68-69), and the genotyping markers for strain confirmation (Sme16S, SEQ ID NOS:33-34, and NodD1, SEQ ID NOS:35-36, or NodQ, SEQ ID NOS:37-38, for Rhizobium and S. meliloti, respectively).

As further evidence of binary vector maintenance in these strains, plasmid DNA is prepared from cultures grown for 2 d at 28° C. with or without selection (Km50+Sp100). The plasmid DNA, typically digested with one or more restriction enzymes, is separated on 1.2% agarose. The binary vector is visible in all preps.

In a further experiment, the Ti plasmid pTi1 is mobilized from the Agrobacterium strain EHA105 containing pTi1 and RP4-4 to the Bradyrhizobium japonicum strain USDA110 in a biparental mating, followed by selection on YM with Rif100 (for B. japonicum) and Km50 and Cb100 (for pTi1). A colony of B. japonicum is obtained that contained pTi1. This strain is then electroporated with pCAMBIA1105.1R.

Using a Rhizobium spp. NGR234 strain containing pTi1 and RP4-4, the pTi1 is also mobilized to Mesorhizobium loti MAFF303099 in a biparental mating overnight. The M. loti strain is first modified by transposon insertion of a single copy gentimicin resistance gene (confirmed by Southern blotting); selection of transconjugants was done on YM with Gm30 (for M. loti) and Km50 (for pTi1). Several dozen M loti transconjugants are obtained that contain pTi1. Most of these also acquire RP4-4; screening by amplification is therefore done on 80 transconjugant colonies and 3 colonies are identified that did not contain RP4-4. One of these strains is then electroporated with pCAMBIA1105.1R.

Plant tissue is then transformed. Successful transformation is verified by staining for GUS activity. As a positive control, an Agrobacterium donor strain is transformed with the related vector pCAMBIA1105.1 or pCAMBIA1405.1 and used to transform plant cells.

In another experiment, the gene transfer competent S. meliloti strains have retained the ability for nodulation of alfalfa. Alfalfa seeds were germinated, brought into contact with S. meliloti and grown for 4 weeks in large Petri dishes with growth medium. Nodules formed on the roots of plants inoculated with both the wildtype strain and the engineered strains of S. meliloti, indicating that the presence of the Ti plasmid and binary vector did not impair nodulation.

### Example 7

### Rhizobium-Mediated Transformation of Rice

Plant material: Surface-sterilized rice seeds are grown on 2N6 medium containing auxin (2,4-D) in darkness at 26° C. for three weeks (21 d) to form calluses. Scutellum-derived calli obtained from these seeds are used for transformation.

Bacterial strains: In this example, rice calli are transformed with the Rhizobium spp. NGR234 and S. meliloti 1021, both harboring pTi3 and pCAMBIA1105.1R (see Examples 4 and 5 for the construction of these strains).

Control strains: Agrobacterium strain EHA105 that harbors the pCAMBIA1405.1 vector is used for transformation. The vir helper Ti plasmid in strain EHA105 (Hood et al., Transgenic Res. 2:208-218, 1993) is derived from succinamopine type supervirulent Ti plasmid pTiBo542.

Protocol: Day 1: After three weeks of callusing, scutellum-derived calli are subdivided into 4 to 8 mm diameter pieces and placed on plates containing 2N6 medium and incubated at 26° C. in the dark for four to seven days.

Day 2/3: Rhizobia strains are streaked on YM medium with appropriate antibiotics (Km40 and Spec80) and incubated at 29° C. for three days. At this time, the cells form a lawn on the plates. Agrobacterium strains are streaked on AB medium containing Kan50 and Spec100, and grown for two days at 29° C. Extreme care is taken not to contaminate the rhizobial cultures with Agrobacterium.

Day 5: The bacteria are resuspended in AAM or minA medium containing 100 μM acetosyringone (AS) by scraping the bacteria from the plates with an inoculation loop. The OD of the bacterial suspension is measured at 600 nm, and adjusted to an OD of 1.0 for Agrobacterium and 1.5 for the rhizobia (corresponding to mid-exponential growth phase). The suspensions are incubated at room temperature for 3 h. Then, 20 mL of the bacterial suspension is transferred into a Petri dish or other suitable sterile container. Four to seven-day incubated calli are added to the bacterial suspension, swirled and left for 30 min. The calli are then blotted dry on sterile Whatman No. 1 filter papers and transferred to 2N6-AS plates. The calli are co-cultivated for 3 to 5 days in the dark at 26° C. In one embodiment, the suspension and co-cultivation media used for the rhizobia strains are modified in order to provide sufficient support for gene transfer to happen. For example, S. meliloti requires biotin for growth, which may be added to the medium. Similarly, both rhizobial strains show poor growth on 2N6-AS medium; growth improvement, and likewise, an increase in transformation is seen on RMOB medium (used for tobacco, see Example 8) containing 100 μM AS and 5 μg/l biotin.

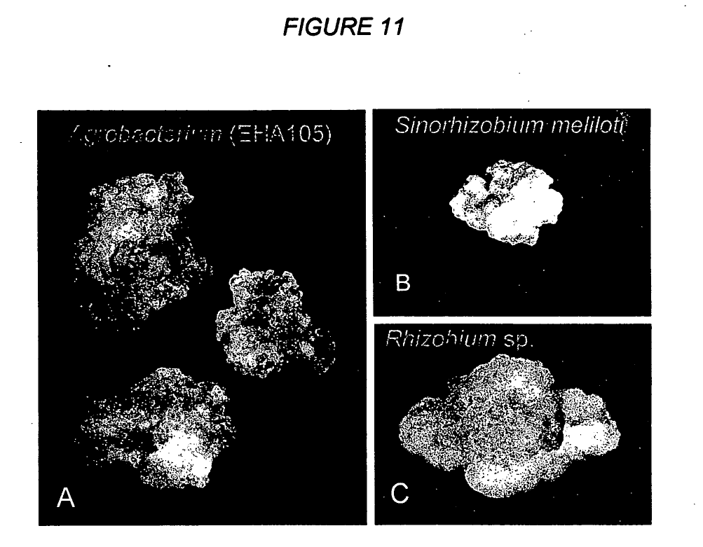

Day 7: Calli co-cultivated with bacteria are washed with water containing 250 mg/L cefotaxime to remove the bacteria; this is done by transferring the calli to plates containing 25 mL of water supplemented with 250 mg/L cefotaxime, swirling, and incubating for 20 min. During this period most of the bacteria are released from the calli. The calli are blotted dry on sterile Whatman No. 1 filter paper and then transferred to 2N6-CH plates containing cefotaxime at 250 mg/L (to kill bacteria left attached to the calli) and hygromycin at 50 mg/L (to select for transgenic calli). Calli are incubated in the dark at 26° C. Transient GUS expression is tested by staining a few washed calli with X-gluc (5-Bromo-4-chloro-3-indolyl β-D glucuronide). FIG. 11 shows calli stained for GUS activity following a five day co-cultivation with Agrobacterium, Sinorhizobium or Rhizobium spp. strains. Blue stained zones are observed on the calli following co-cultivation with rhizobia, though at a lower frequency compared to those observed following co-cultivation with Agrobacterium.

The calli are transferred to fresh selection medium once every two weeks. Small, transgenic hygromycin-resistant calli start proliferating after four weeks of selection on hygromycin. The proliferated calli are sub-cultured and independent proliferating lines are made. These sub-cultured calli further proliferate within two weeks and are transferred to regeneration medium and cultured in the dark for one week.

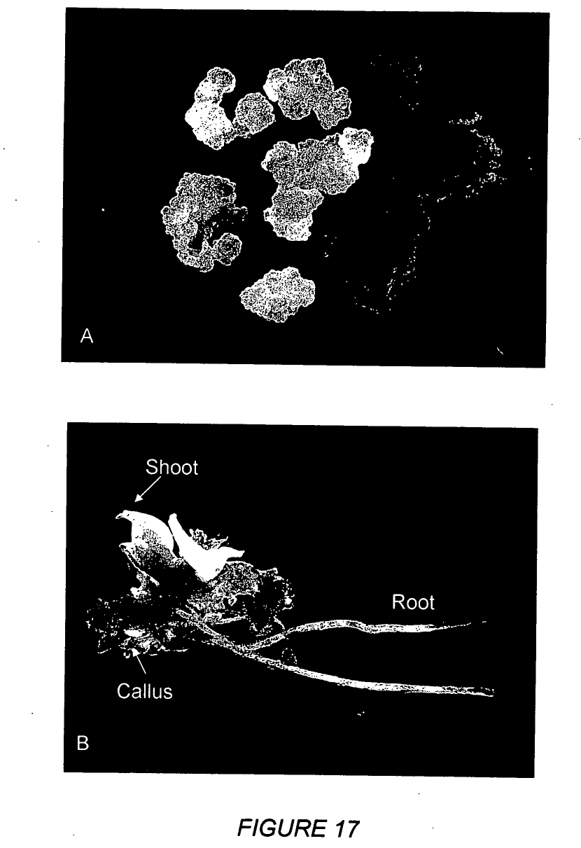

After a week, the calli are transferred to light. Five to ten days later calli start turning green and in two to three weeks time shoots start differentiating. These shoots are then transferred onto rooting medium, and once roots are formed, plants are hardened and transferred to the glass house. FIG. 17 shows a GUS stained rice plantlet obtained after co-cultivation with S. meliloti containing pTi3 and pCAMBIA1105.1R. GUS expression is observed in the root, at the base of the shoot, and in the leaf tip. Amplification analysis revealed the presence of the pCAMBIA1105.1R-specific MCS, confirming that the T-DNA integrated in this plant originated from the S. meliloti strain.

### Example 8

### Rhizobia-Mediated Transformation of Tobacco

In this example, tobacco leaf discs are transformed by rhizobia containing a Ti plasmid and binary vector. The explant tissues used in this experiment are 1 cm² leaf discs punched out of the upper expanded tobacco leaf from a four-five week old tissue culture grown rooted plant. The bacteria used in this example are Rhizobium spp. NGR234 (ANU240) and S. meliloti 1021, both containing pTi3 and pCAMBIA1105.1R (see Examples 3 to 5). As a positive control for gene transfer, the Agrobacterium EHA105 strain containing pTi1 and pCAMBIA1405.1 is used.

Day 1: Bacteria are plated out onto YM plates with Kan40 and Spec80 (rhizobia) or minA plates with Km50 and Spec100 (Agrobacterium). Plates are incubated at 28 C for two to three days.

Day 4: The bacteria are scraped of the plates and resuspended in 20 mL of minA liquid up to an OD at 600 nm of 1.0 to 1.5. Leaf discs are cut out of the upper tobacco leaf, transferred to a Petri dish containing the bacterial suspension, and incubated for 5 min. Discs are blotted dry on Whatman no. 1 filter paper and placed upside down on solid RMOP co-cultivation medium. Plates are incubated for two (Agrobacterium) or five to seven days (rhizobia) in the dark at 28 C.

Day 6/9: Leaf discs are transferred to selection plates (RMOP-TCH) and incubated two-three weeks in the light at 28° C. with 16 hr daylight per day. Subculture leaf discs every two weeks. When shoots appear, the plantlets are transferred to MST-TCH plates for plantlet regeneration. If roots appear, the plantlets are transferred to soil in the glasshouse.

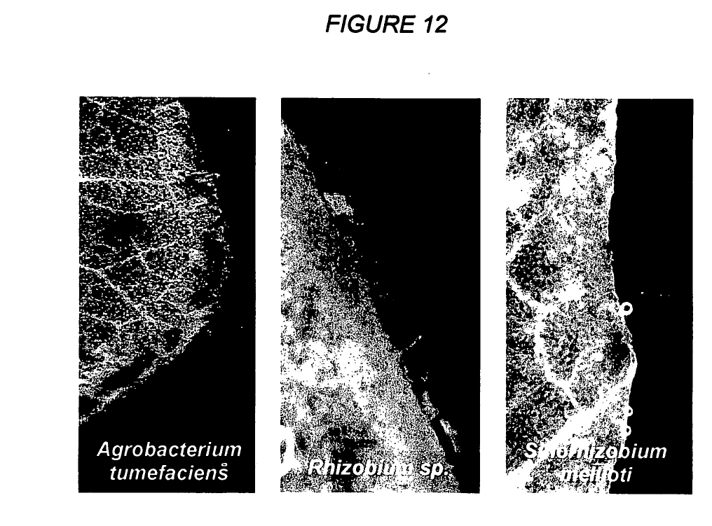

Gene transfer efficiency is monitored immediately after co-cultivation by staining the leaf discs in X-gluc overnight (Jefferson, Plant Mol. Biol. Rep 5:387-405, 1987). Table 2 shows the results of a typical tobacco transformation experiment using both rhizobia strains and the Agrobacterium strain as a control. FIG. 12 shows a few images of tobacco leaves transformed with these bacteria.

TABLE 2

| | | | | | Average |
|---|---|---|---|---|---|
| | | | No. of | Total | no. blue |
| | | | leaf | no. | spots |
| Bacterial | Ti | | disks | of blue | per |
| species | plasmid | Binary vector | assayed | spots | disk |
| Rhizobium spp. | pTi3 | pCambia1105.1R | 10 | 2 | 0.2 |
| Sinorhizobium | pTi3 | pCambia1105.1R | 10 | 59 | 6 |
| meliloti | | | | | |
| Agrobacterium | pTi1 | pCambia1405.1 | 10 | ˜3000 | ˜300 |
| tumefaciens | | | | | |

Table 3 shows the result of several transformation experiments using S. meliloti with pTi3 and pC1105.1R. The use of younger tobacco leaves increased gene transfer dramatically (15× more blue spots per leaf disk compared to slightly older leaves); for Agrobacterium-mediated transformation, gene transfer appears more or less similar for both leaf types.

TABLE 3

| | | | Number of | Average blue |
|---|---|---|---|---|
| Bacterial | Ti | | leaf disks | spots per |
| species | plasmid | Binary vector | assayed | disk |
| Sinorhizobium | pTiWB3 | pC1105.1R | 10 | 5.9 |
| meliloti | | | | |
| Sinorhizobium | pTiWB3 | pC1105.1R | 10 | 6.3 |
| meliloti | | | | |
| Sinorhizobium | pTiWB3 | pC1105.1R | 10 | 2.2 |
| meliloti | | (old leaf material) | | |
| Sinorhizobium | pTiWB3 | pC1105.1R | 10 | 30.6 |
| meliloti | | (young leaf | | |
| | | material) | | |

In order to ascertain that the rhizobia cultures used for tobacco leaf treatment are free of any contaminating Agrobacterium cells, the bacterial suspensions used for leaf treatment are plated out on media that favor the growth of Agrobacterium colonies in comparison with that of the non-Agrobacteria; Rhizobium cannot grow on LB plates, while Agrobacterium does and S. meliloti requires the inclusion of biotin in minimal media which Agrobacterium is not dependent on. In a typical assay, 100 μl of the bacterial suspension is plated out onto a single plate and incubated at 28° C. for five days. No bacterial colonies are observed on these plates, indicating that there are potentially less than 200 Agrobacterium cells present in the (20 ml) suspension used for explant treatment. The presence of even 1000 Agrobacterium cells harboring pC1305.1 in a 20 mL suspension of S. meliloti containing pTi3 but without binary vector (Sme pTi3) does result in only a few blue spots in and add-back experiment, the results of which are shown in Table 4.

TABLE 4

| | Total number | Total number | | |
|---|---|---|---|---|
| | of Sme pTi3 | of EHA105 | No. leaf | |
| Treatment | cells | cells | disks | GUS activity |
| 1 | 10¹⁰ | 0 | 10 | No GUS activity |
| 2 | 10¹⁰ | 10² | 10 | 1 blue spot |
| 3 | 10¹⁰ | 10³ | 10 | 3 blue spots (1 |
| | | | | spot on each of |
| | | | | three disks) |
| 4 | 10¹⁰ | 10⁵ | 10 | 423 blue spots |
| | | | | (42 spots/disk) |
| 5 | 0 | 10¹⁰ | 9 | 300-400 blue |
| | | | | spots per disk |

As further proof that Agrobacterium is absent in the tobacco transformation experiment, the bacterial mass that has grown on the co-cultivation plates is washed of the plates after removal of the explants by the addition of 2 mL of LB medium to the plates and shaking for 1 h at 28° C. Then 100 μl of this suspension is plated onto plates favoring Agrobacterium growth. Again, no colonies are growing on these plates in a typical experiment. Furthermore, 100 μl of the bacterial suspensions before and after co-cultivation are spun down, resuspended in sterile water and used for amplification analysis using the Agrobacterium-specific attScirc primers (SEQ ID NOS:23-24) and the Sme16S primers (SEQ ID NOS:33-34) as a positive control. The results confirm absence of Agrobacterium DNA in the samples.

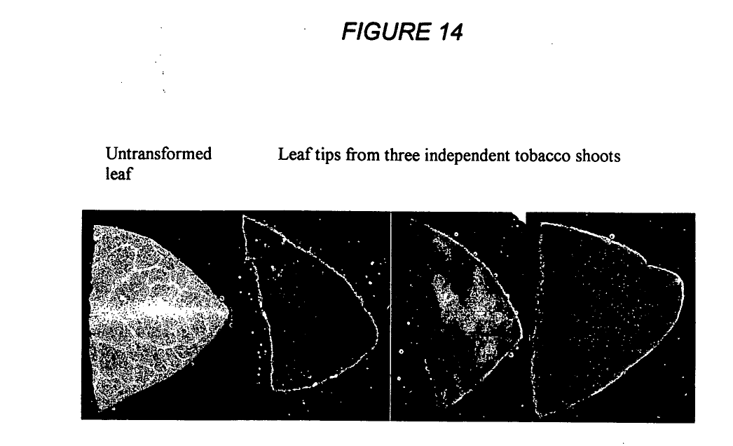

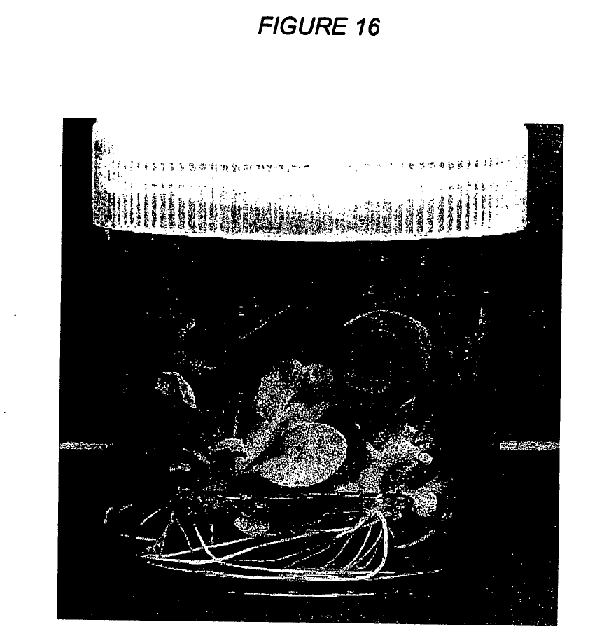

Leaf disks co-cultivated with S. meliloti pTi3 pC105.1R and with Agrobacterium pTi1 pC1405.1 are cultured on regeneration medium containing hygromycin. Shoots are developed and plantlets regenerated. FIG. 16 shows a picture of tobacco plants regenerated following co-cultivation with the gene transfer proficient S. meliloti strain. The leaf tip from a number of independent plants is stained for GUS activity. The result is shown in FIG. 14, revealing strong GUS activity in each of three leaf tips assayed while an untransformed tobacco leaf tip shows no blue staining. Table 5 shows the number of rooted plants regenerated following two independent transformation experiments with S. meliloti pTi3 pC1105.1R and A. tumefaciens pTi1 pC1405.1. The formation of roots by shoots cultured on media containing selection (50 mg/L hygromycin) is a good indication that the shoot is genetically transformed. The data are an underestimate of root formation as the data were collected at an early time point and some of these shoots may still form roots. As shown in the table below, the number of putatively transformed shoots recovered per leaf disk is only 5 to 9 times lower for S. meliloti-mediated transformation compared to Agrobacterium-mediated transformation.

TABLE 5

| | | No. leaf | | No. | No. |
|---|---|---|---|---|---|
| | | disks | No. | shoots | transformed |
| Bacterial | Experi- | co- | shoots | forming | shoots/ |
| species | ment | cultured | collected | roots* | leaf disk |
| S. meliloti | 30.04.04 | 20 | 9 | 2 (22%) | 2/20 (10%) |
| S. meliloti | 16.04.04 | 34 | 24 | 6 (25%) | 6/34 (18%) |
| A. tumefaciens | 16.04.04 | 10 | 48 | 9 (19%) | 9/10 (90%) |

The plants regenerated from the leaf discs are analyzed by amplification of the T-DNA markers. Genomic DNA is isolated from a leaf piece and used for amplification of the hygromycin gene (SEQ ID NOS:82-83) and the MCS sequence (SEQ ID NO:46 and 79). The results are shown in FIG. 15 and are summarized in Table 6. All four plants co-cultivated with S. meliloti and all three plants co-cultivated with A. tumefaciens show the presence of the hygromycin band and are thus confirmed to be transformed. Moreover, all four S. meliloti-transformed plants reveal a 570 bp amplification product, consistent with the corresponding sequence in pCAMBIA1105.1R; in contrast, the Agrobacterium-transformed plants reveal the 490 bp product, corresponding to the MCS sequence in pC1405.1. This result confirms the presence in the S. meliloti-transformed plants of the T-DNA region derived from the rhizobia-specific marked pCAMBA1105.1R vector and not from pCAMBIA1405.1, which has a smaller MCS and has been electroporated to Agrobacterium strains only.

TABLE 6

| Plant | Co-culture | Binary | GUS | | MC site |
|---|---|---|---|---|---|
| Number | Bacterium | Vector | activity | HygR | (491 or 572 bp) |
| 2-1 | S. meliloti | pC1105.1R | Yes | + | + (572) |
| 6 | S. meliloti | pC1105.1R | Yes | + | + (572) |
| 7-1 | S. meliloti | pC1105.1R | No | + | + (572) |
| 11-1 | S. meliloti | pC1105.1R | Yes | + | + (572) |
| 1 | A. tumefaciens | pC1405.1 | Yes | + | + (491) |
| 2 | A. tumefaciens | pC1405.1 | Yes | + | + (491) |
| 3 | A. tumefaciens | pC1405.1 | Yes | + | + (491) |

| Non-transgenic Wisconsin 38 | | No | − | − |
|---|---|---|---|---|
| | Plasmid pC1405.1 | | + | + (491) |
| | Plasmid pC1105.1R | | + | + (572) |
| | No DNA control | | − | − |

Similarly, five tobacco plants are obtained following co-cultivation with Rhizobium spp. NGR234 containing pTi3 and pCAMBIA1105.1R. All these express GUS in their leaves and reveal the expected amplification bands for the MCS and HygR gene, confirming that they result from Rhizobium-mediated transformation.

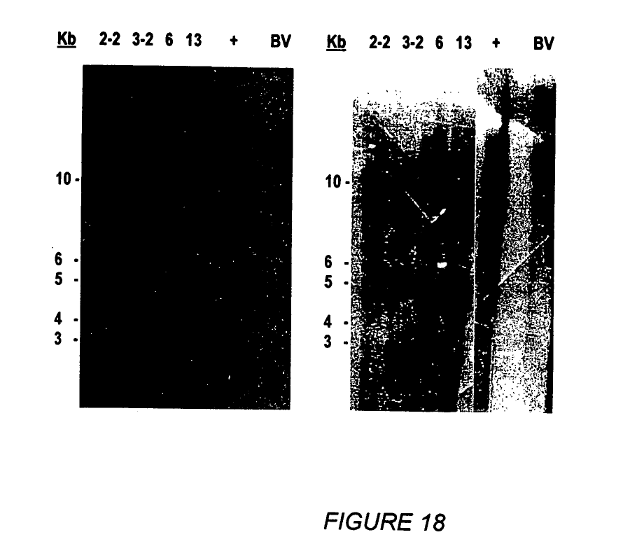

Four tobacco plants are subjected to Southern blot transfer and hybridization. FIG. 18 shows the hybridization pattern of restricted genomic DNA from four transformants (2-2; 3-2; 6; and 13), a transformed rice plant that contains a single copy (+), and pC1105.1R vector DNA (BV) in an amount equivalent to single copy integrant. The blot is probed with labeled DNA from a hygromycin gene (left panel), stripped, and probed with labeled DNA from GUSplus gene (β-glucuronidase from Staphylococcus). They hybridization patterns differ for each transformant, evidencing that each plant is the result of an independent transformation.

Tobacco leaf discs are co-cultivated with Mesorhizobium loti constructed as in Example 6. After five days of co-cultivation, four areas stain positive for GUS expression on a total of 10 leaf discs; after seven or nine days co-cultivation, respectively 55 and 25 GUS-expressing foci are seen on 10 leaf discs each.

### Example 9

### Effect of RP4 Presence on Gene Transfer

Gene transfer to plants following T-DNA excision and transfer has many similarities with bacterial conjugation (e.g. Pansegrau et al., Proc. Natl. Acad. Sci USA 90:11538-11542, 1993; Hamilton et al., J. Bacteriol. 154:693-701, 2000; Bravo-Angel et al., J. Bacteriol. 181:5758-5765, 1999). Moreover, some mobilizable plasmids such as RSF1010 and CloDF13 can be transferred to plant cells by the virB system of the Ti plasmid (Fullner, J. Bacteriol. 180:430-434, 1998; Escudero et al., Mol. Microbiol. 47:891-901, 2003), and transformed plants have been obtained by Agrobacterium-mediated transformation with a GUS containing pClo vector without the T-DNA borders (Escudero et al., Mol. Microbiol. 47:891-901, 2003). Furthermore, the presence of RSF1010 in wildtype Agrobacterium strains inhibits their virulence by a process in which the transferred form of the plasmid competes with the virD2-T strand complex and/or virE2 for a common export site (Stahl et al., J. Bacteriol. 180:3933-3939, 1998). Here we show that the presence of RP4-4, a kan-sensitive derivative of the broad-host range IncP plasmid RP4, in gene transfer competent bacteria, interferes with their capacity for gene transfer to plants.

Tobacco leaf disks and rice calli are co-cultivated with bacterial strains containing a Ti plasmid and binary vector and with or without the RP4-4 plasmid. Strains containing RP4-4 are made by conjugative transfer of the plasmid from E. coli containing RP4-4 and selecting the transconjugants on carbenicillin100. Alternatively, RP4-4 containing strains may be selected among the population of bacteria that are obtained following conjugation of the modified Ti plasmid from EHA105 to any of the rhizobial strains, using the E. coli RP4-4 strain as a helper strain. The presence or absence of RP4-4 in the strains is confirmed by amplification in the presence of primers for part of the RP4 plasmid (SEQ ID NOS:80-81), using an annealing temperature of 62 degrees to prevent nonspecific binding. In this example, the gene transfer capacity is assessed for Agrobacterium strain EHA105 containing pC1405.1 with and without RP4-4. The results are summarized in Table 7. In the absence of RP4-4, approximately 3000 GUS-expressing blue spots are detected on 10 tobacco leaf disks assayed. In contrast, the strain that contains the RP4-4 plasmid yielded only 73 blue spots for 10 disks, which is only 2.4% of the gene transfer efficiency of the RP4-4-less strain. In rice calli transformation, the result is even more pronounced: no GUS activity is observed in 93 calli following co-cultivation with the RP4-4 containing Agrobacterium strain, while 27 out of 30 calli stained showed GUS activity. This indicates that the presence of the RP4-4 plasmid hampers gene transfer, possibly by the interference of some part of the conjugation process with T-DNA or vir protein transfer to plant cells.

In a similar experiment using the S. meliloti and Rhizobium spp. NGR234 strains harboring a Ti plasmid and binary vector, the above result was confirmed (see Table 7). Tobacco co-cultivation with the S. meliloti strain containing RP4-4 produced no GUS expressing spots on 10 leaf disks tested, while a similar strain devoid of RP4-4 produced 22 and 306 blue spots on 10 disks each for older and younger leaf material respectively. For the RP4-4-less Rhizobium spp. strain, 2 blue spots were seen, while no spots were obtained for the RP4-4 containing strain of the same species. Again, the result suggests a profound negative effect of the IncP plasmid on the transformation ability of the strains.

TABLE 7

| | | | | |
|---|---|---|---|---|
| Bacterial | Ti Plasmid + | Binary | No. disks | |
| species | RP4-4 | vector | assayed | GUS Activity |

TOBACCO

| | | | | |
|---|---|---|---|---|
| A. tumefaciens | pEHA105 + | 1405.1 | 10 | 73 spots total |
| | RP4-4 | | | |
| A. tumefaciens | pEHA105 | 1405.1 | 10 | ˜3000 spots |
| | | | | total |
| S. meliloti | pTiWB1 + | 1105.1R | 10 | None |
| | RP4-4 | | | |
| S. meliloti | pTiWB3 | 1105.1R | 10 (old) | 22 spots total |
| S. meliloti | pTiWB3 | 1105.1R | 10 (young) | 306 spots total |
| Rhizobium spp. | pTiWB1 + | 1105.1R | 10 | None |
| NGR234 | RP4-4 | | | |
| Rhizobium spp. | pTiWB3 | 1105.1R | 10 | 2 spots on 1 |
| NGR234 | | | | disk |

RICE

| | | | | |
|---|---|---|---|---|
| A. tumefaciens | pEHA105 | 1405.1 | 30 calli | 27/30 calli |
| | | | | show activity |
| A. tumefaciens | pEHA105 + | 1405.1 | 93 calli | None |
| | RP4-4 | | | |

### Example 10

### Rhizobia-Mediated Transformation of Arabidopsis Flower Tissues

Arabidopsis is transformed by Rhizobium containing a Ti plasmid and a binary vector using the commonly used floral dip method (Clough and Bent, Plant J. 16:735-743, 1998). The immature floral stems of potted Arabidopsis plants are dipped into a bacterial suspension, flowering and seed formation is allowed to proceed and the seeds are harvested and germinated onto media selective for the growth of the transformants. The bacteria used in this example are Rhizobium spp. NGR234 (ANU240) and S. meliloti 1021, both containing pTi3 and pCAMBIA1105.1R (see Examples 3 to 5). As a positive control for gene transfer, the Agrobacterium EHA105 strain containing pTi3 and pCAMBIA1405.1 is used.

Arabidopsis seeds are surface sterilized in 70% ethanol and then in 20% hydrogen peroxide+0.02% Triton X-100 and germinated in Petri dishes containing Arabidopsis germination medium (AGM). Germinated seedlings are individually transferred to soil and incubated in a growth room at 26° C. for several weeks until they start to flower.

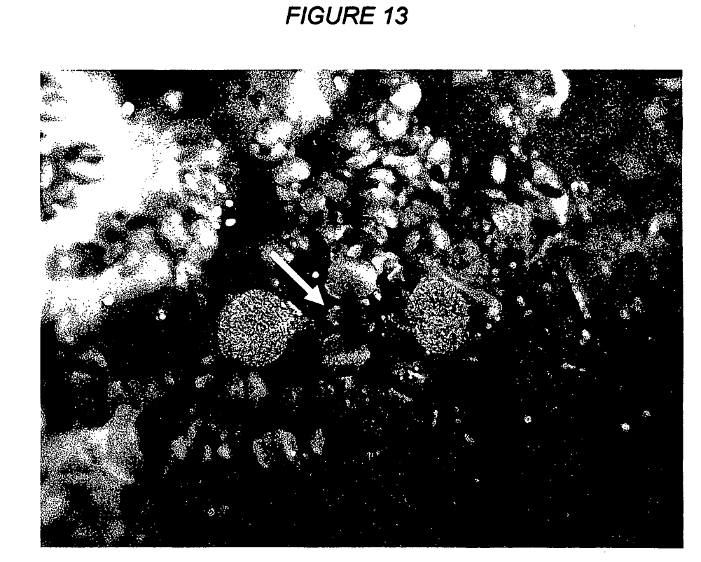

Bacteria are plated out onto YM plates with Kan40 and Spec80 (rhizobia) or minA plates with Km50 and Spec100 (Agrobacterium). Plates are incubated at 28° C. for two to three days. Bacteria are resuspended from the plates in Infiltration Medium (1×MS salts, 5% sucrose, 50 mM MES-KOH pH 5.7, 0.1% Silwet L-77) to give an OD at 600 nm of 1.0. The inflorescences are dipped into the bacterial suspension. The plants are covered to maintain a high humidity overnight and grown thereafter uncovered at 20° C. Seeds are harvested, surface sterilized as described above and germinated on plates containing 1×MS salts, 3% sucrose, 0.05% MES-KOH pH5.7, 0.8% Phytagel and hygromycin. at 30 μg/mL. putative transformants are plated to soil. At this stage, leaves may be stained for GUS activity to assay the presence of the T-DNA. FIG. 13 shows the results of a transformation experiment using the Rhizobium spp. strain. In this experiment, 1 out of 300 seeds was hygromycin-resistant. The result shows that Rhizobium spp. NGR234 can transform Arabidopsis flowers by floral dip transformation. In a similar experiment, the S. meliloti strain containing pTi3 and pCAMBIA1105.1R yielded 3 hygromycin-resistant Arabidopsis seedlings that expressed GUS and had integrated the pCAMBIA1105.1R-specific MCS and HygR marker as revealed by amplification.

### Example 11

### Rhizobia-Mediated Whole Plant Transformation

Plant transformation protocols have largely been developed for Agrobacterium-mediated transformation. Using the bacteria of this invention, which interact with plants and plant tissues in a different way, both the protocols and the tissues that are used for transformation are modified in order to accommodate the specific characteristics of the bacteria-plant interactions. In this example, rhizobial species containing a pTi and binary vector are used for whole plant transformation of the common bean (Phaseolus sativa). The bacteria used in this example are the strains Rhizobium spp. NGR234 (ANU240) and S. meliloti 1021, both containing pTi3 and pCAMBIA1105.1R. Cells growing in liquid TY medium with Km40 and Sp80 up to an OD at 600 nm of 1.5 are pelleted, resuspended in AAM medium with 100 μM acetosyringone and used for plant co-cultivation.

Beans are surface sterilized and germinated on wet filter paper in a Petri dish. The seedlings are incubated in the bacterial suspension for 30 min, blotted dry and transferred to wet filter paper. After 5 days co-cultivation, the seedlings are stained for GUS activity by treatment with X-Gluc. Blue spots on a seedling indicate the presence of cells that have acquired and express the GusPlus containing T-DNA.

From the foregoing, it will be appreciated that, although specific embodiments of the invention have been described herein for purposes of illustration, various modifications may be made without deviating from the spirit and scope of the invention. Accordingly, the invention is not to be limited except as by the appended claims.

Table of Sequences

| SEQ | | |
|---|---|---|
| ID | | |
| NO. | Name | Sequence 5′-3′ |
| 1 | 16S rDNA Rhizobium | see FIG. 2 |
| | spp. NGR234 | |
| 2 | atpD Rhizobium spp. | see FIG. 2 |
| | NGR234 | |
| 3 | recA Rhizobium·spp. | see FIG. 2 |
| | NGR234 | |
| 4 | 16S rDNA | see FIG. 2 |
| | S. meliloti 1021 | |
| 5 | atpD S. meliloti | see FIG. 2 |
| | 1021 | |
| 6 | recA S. meliloti | see FIG. 2 |
| | 1021 | |
| 7 | 165 rDNA M loti | see FIG. 2 |
| | MAFF303099 | |
| 8 | atpD M loti | see FIG. 2 |
| | MAFF303099 | |
| 9 | recA M loti | see FIG. 2 |
| | MAFF303099 | |
| 10 | 16S rDNA | see FIG. 2 |
| | P. myrsinacearum | |
| 11 | atpD | see FIG. 2 |
| | P. myrsinacearum | |
| 12 | 16S rDNA | see FIG. 2 |
| | B.japonicum | |
| | USDA1 10 | |
| 13 | atpD B.japonicum | see FIG. 2 |
| | USDA110 | |
| 14 | recA B.japonicum | see FIG. 2 |
| | USDA110 | |
| 15 | 16S rDNA | see FIG. 2 |
| | A. tumefaciens | |
| | EHA105 | |
| 16 | atpD A. tumefaciens | see FIG. 2 |
| | EHA1O5 | |
| 17 | recA A. tumefaciens | see FIG. 2 |
| | EHA10S | |
| 18 | Rle16Sfw | CACGTAGGCGGATCGATC |
| 19 | Rle16Srev | TTAGCTCACACTCGCGTGCT |
| 20 | Atu16Sfw | GGCTTAACACATGCAAGTCGAAC |
| 21 | Atu16Srev | CGGGGCTTCTTCTCCGACT |
| 22 | Atu16Sfw2 | GAATAGCTCTGGGAAACTGGAAT |
| 23 | AttScircfw | CAGGCTCAAACCGCATTTCC |
| 24 | AttScircrev | GTAAGTCCAGCCTCTTTCTCA |
| 25 | AttSpATfw | GTGCTTCGGATCGACGAAAC |
| 26 | AttSpATrev | GGAGAATGGGAGTGACCTGA |
| 27 | AtuvirGfw | CGCTAAGCCGTTTAGTACGA |
| 28 | AtuvirGrev | CCCCTCACCAAATATTGAGTGTAG |
| 29 | NptIfw | CAGGTGCGACAATCTATCGA |
| 30 | NptIrev | AGCCGTTTCTGTAATGAAGG |
| 31 | VirBfw | TGACCTTGGCCAGGGAATTG |
| 32 | VirBrev | TCCTGTCATTGGCGTCAGTT |
| 33 | Sme16Sfw | TGTGCTAATACCGTATGAGC |
| 34 | Sme16Srev | CAGCCGAACTGAAGGATACG |
| 35 | NodD1NGR234fw | GCCAGAAATGTTCATGTCGCACA |
| 36 | NodD1NGR234rev | AATGGGTTGCGGAAGTTCGGT |
| 37 | SmeNodQfw | GACAGGATCCTCCACGCTCA |
| 38 | SmeNodQrev | CGCCAGGTCGTTCGGTTGG |
| 39 | SmeNodQ2rev | GCTCATAGGGCGAGGATACA |
| 40 | VirB11FW2 | ACGGCGCGAATCCAATCCAA |
| 41 | M13REV | CAGGAAACAGCTATGAC |
| 42 | M13FW | GTAAAACGACGGCCAG |
| 43 | MoaArev2 | TAAGCGTCCCATCGAGATCG |
| 44 | HygRfw | GCATCTCCCGCCGTGCACAG |
| 45 | HygRrev | GATGCCTCCGCTCGAAGTAGCG |
| 46 | 1405.1fw | CTGGCACGACAGGTTTC |
| 47 | 16Sfw63 | CAGGCTTAACACATGCAAGTC |
| 48 | 16Srev801 | ACCAGGGTATCTAATCCTGT |
| 49 | 16Sfw714 | GAACACCAGTGGCGAAGGC |
| 50 | 16Srev1492 | CGGCTACCTTGTTACGACTT |
| 51 | atpDfw294 | ATCGGCGAGCCGGTCGACGA |
| 52 | AtpDrev771 | GCCGACACTTCCGAACCNGCCTG |
| 53 | recAfw63 | ATCGAGCGGTCGTTCGGCAAGGG |
| 54 | RecArev504 | TTGCGCAGCGCCTGGCTCAT |
| 55 | Mlo16Sfw | CCCATCTCTACGGAACAACT |
| 56 | Mlo16Srev | ACTCACCTCTTCCGGACTCG |
| 57 | MlopMLaRepCfw | GACGGCCGAGCCAAGGACGA |
| 58 | MlopMLRepCrev | CACATGGCAAGCCTCCTCA |
| 59 | MlopMlbrepCfw | GATGCTGGAAAGCTTCACAAGT |
| 60 | Pmy16Sfw | CTGGTAGTCTTGAGTTCGAG |
| 61 | Pmy16Srev | CCAGCCTAACTGAAGGAAAC |
| 62 | PmyGyrBfw | CTGGCTGCGTCTCAAGATTC |
| 63 | PmyGyrBrev | CCTTTGCCTTCTTCGCCTGC |
| 64 | Bja16Sfw | GGGCGTAGCAATACGTCA |
| 65 | Bja16Srev | CTTCGCCACTGGTGTTCTTG |
| 66 | VirB11fw | ATAAGCTTCTCTACGGCGATCGAT |
| | | GTCA |
| 67 | VirC2rev | ATCTGCAGTGCTCGAGGTCGCTCG |
| | | AAGT |
| 68 | MoaAfw | ATGGATCCGGTCTTGAAAGCTTGG |
| | | CTCA |
| 69 | MoaArev | ATGGATCCTGCCGTGGTCTCGTGT |
| | | TCTGG |
| 70 | AccAfw | ATGGATCCGAGCAGGGAGAGGACA |
| | | ACCA |
| 71 | AccArev | ATGGATCCTCGGGTCCTGAAAGAT |
| | | CATC |
| 72 | OriTfw | GGATCCTCTAGACTGGAAGGCAGT |
| | | ACACCTTGATAG |
| 73 | OriTrev | GGATCCTCTAGATTCCTGCATTTG |
| | | CCTGTTTCCAG |
| 74 | AccAfw2 | AGCTGCGGAAGAAGCTCGT |
| 75 | MoaArev2 | TAAGCGTCCCATCGAGATCG |
| 76 | SpecfwNsiI | ATGCATGATATATCTCCCAATTTGTG |
| 77 | SpecrevSacII | CCGCGGATGACAGAGCGTTGCTGCC |
| | | TGTGATCAATT |
| 78 | SpecfwSacII | CCGCGGCATGATATATCTCCCAATTT |
| 79 | P35S5′rev | TACGGCGAGTTCTGTTAGGT |
| 80 | RP4fw | AGCTGGCTGACGAACCTGCG |
| 81 | RP4rev | GGCGTCCTTGGAACGATGCT |
| 82 | Hyg700fw | ACTCACCGCGACGTCTGTC |
| 83 | Hyg700rev | GCGCGTCTGCTGCTCCAT |

1. A method for the introducing a DNA sequence of interest into plants, comprising: contacting a plant or a plant tissue or a plant cell or a protoplast with non-pathogenic Rhizobium spp., Sinorhizobium meliloti, Mesorhizobium loti, Phyllobacterium myrsinacearum, or Bradyrhizobium japonicum bacteria that contain:
(i) a first plasmid comprising a vir gene region of a Ti plasmid, and
(ii) a second plasmid comprising one or more T-border sequences operatively linked to a DNA sequence of interest;
wherein the products of the vir genes act to introduce the DNA sequence of interest into the plant, plant tissue, plant cell or protoplast.

2. The bacteria of claim 1, wherein the first plasmid is a disarmed Ti plasmid from Agrobacterium.

3. The method of claim 1, wherein the first plasmid or the second plasmid or both plasmids further comprise a sequence encoding a selectable product.

4. The method of claim 1, wherein the sequence encoding the selectable product of the second plasmid is operatively linked to the T-border sequences and the product can be selected for in plants.

5. A method for the introducing a DNA sequence of interest into plants, comprising: contacting a plant or a plant tissue or a plant cell or a protoplast with non-pathogenic Rhizobium spp., Sinorhizobium meliloti, Mesorhizobium loti, Phyllobacterium myrsinacearum, or Bradyrhizobium japonicum bacteria that contain:
(i) a first nucleic acid molecule comprising a vir gene region of a Ti plasmid, and
(ii) a second nucleic acid molecule comprising one or more T-border sequences operatively linked to a DNA sequence of interest;
wherein the products of the vir genes act to introduce the DNA sequence of interest into the plant, plant tissue, plant cell or protoplast.

6. The method of claim 5, wherein the first nucleic acid molecule is integrated into the genome of the non-pathogenic bacteria.

7. The method of claim 5, wherein the first and the second nucleic acid molecules are self-replicating plasmids.

8. The method of claim 5, wherein the first plasmid or the second plasmid or both plasmids further comprise a sequence encoding a selectable product.

9. The method of claim 8, wherein the sequence encoding the selectable product of the second plasmid is operatively linked to the T-border sequences and the product can be selected for in plants.

10. A method for the introducing a DNA sequence of interest into plants, comprising: contacting a plant or a plant tissue or a plant cell or a protoplast with non-pathogenic Rhizobium spp., Sinorhizobium meliloti, Mesorhizobium loti, Phyllobacterium myrsinacearum, or Bradyrhizobium japonicum bacteria that contain:
(i) a first plasmid comprising a vir gene region of a Ti plasmid, and
(ii) a second plasmid comprising one or more oriT sequences of a mobilizable plasmid operatively linked to a DNA sequence of interest;
wherein the products of the vir genes act to introduce the DNA sequence of interest into the plant, plant tissue, plant cell or protoplast.

11. The method of claim 10, wherein the mobilizable plasmid is IncP plasmid RK2, IncP plasmid RP4, IncQ plasmid RSF1010, or IncQ plasmid CloDF13.

12. The method of claim 10, wherein the first plasmid is a disarmed Ti plasmid from Agrobacterium.

13. The method of claim 10, wherein the first plasmid or the second plasmid or both plasmids further comprise a sequence encoding a selectable product.

14. The method of claim 13, wherein the sequence encoding the selectable product of the second plasmid is operatively linked to the T-border sequences and the product can be selected for in plants.

15. A method for the introducing a DNA sequence of interest into plants, comprising: contacting a plant or a plant tissue or a plant cell or a protoplast with non-pathogenic Rhizobium spp., Sinorhizobium meliloti, Mesorhizobium loti, Phyllobacterium myrsinacearum, or Bradyrhizobium japonicum bacteria that contain a nucleic acid molecule comprising a vir gene region of a Ti plasmid and one or more T-border sequences operatively linked to a DNA sequence of interest.

16. The method of claim 15, wherein the nucleic acid molecule is formed by homologous recombination between a vector comprising the T-border sequences and vir gene region and a vector comprising the DNA sequence of interest.

17. Non-pathogenic Rhizobium spp., Sinorhizobium meliloti, Mesorhizobium loti, Phyllobacterium myrsinacearum, or Bradyrhizobium japonicum bacteria that interact with plant cells, comprising:
(a) a first nucleic acid molecule comprising a vir gene region of a Ti plasmid from Agrobacterium, and
(b) a second nucleic acid molecule comprising one or more T-border sequences of a Ti plasmid from Agrobacterium operatively linked to a DNA sequence of interest;
wherein products of the vir genes act to transfer the DNA sequence of interest into the plant, plant cell, plant tissue or protoplast.

18. The bacteria of claim 17, wherein the first nucleic acid molecule is integrated into the genome of the non-pathogenic bacteria.

19. The bacteria of claim 17, wherein the first and the second nucleic acid molecules are self-replicating plasmids.

20. Non-pathogenic Rhizobium spp., Sinorhizobium meliloti, Mesorhizobium loti, Phyllobacterium myrsinacearum, or Bradyrhizobium japonicum bacteria that interact with plant cells, comprising:
(a) a first nucleic acid molecule comprising a vir gene region of a Ti plasmid from Agrobacterium, and
(b) a second nucleic acid molecule comprising one or more oriT sequences of a mobilizable plasmid operatively linked to a DNA sequence of interest;
wherein products of the vir genes act to transfer the DNA sequence of interest into the plant, plant cell, plant tissue or protoplast.

21. The bacteria of claim 20, wherein the mobilizable plasmid is RK2, RP4, RSF1010 or CloDF13.

22. The bacteria of claim 20, wherein the first nucleic acid molecule is integrated into the genome of the non-pathogenic bacteria.

23. The bacteria of claim 20, wherein the first and the second nucleic acid molecules are self-replicating plasmids.

24. Non-pathogenic Rhizobium spp., Sinorhizobium meliloti, Mesorhizobium loti, Phyllobacterium myrsinacearum, or Bradyrhizobium japonicum bacteria that interact with plant cells, comprising:
a first plasmid comprising a vir gene region of a Ti plasmid, and a second plasmid comprising one or more T-border sequences operatively linked to a DNA sequence of interest;
wherein the products of the vir genes act to introduce the DNA sequence of interest into the plant, plant tissue, plant cell or protoplast.

25. The bacteria of claim 24, wherein the first plasmid is a disarmed Ti plasmid from Agrobacterium.

26. The bacteria of claim 24, wherein the first plasmid or the second plasmid or both plasmids further comprises a sequence encoding a selectable product.

27. The bacteria of claim 24, wherein the sequence encoding the selectable product of the second plasmid is operatively linked to the T-border sequences and the product can be selected for in plants.

28. Non-pathogenic Rhizobium spp., Sinorhizobium meliloti, Mesorhizobium loti, Phyllobacterium myrsinacearum, or Bradyrhizobium japonicum bacteria that interact with plant cells that contain a nucleic acid molecule comprising a vir gene region of a Ti plasmid and one or more T-border sequences operatively linked to a DNA sequence of interest.

29. The bacteria of claim 28, wherein the nucleic acid molecule is formed by homologous recombination between a vector comprising the T-border sequences and vir gene region and a vector comprising the DNA sequence of interest.


SEQUENCE LISTING (83 sequences)

SEQ ID NO: 1 - DNA, 1408 bp, Rhizobium spp. NGR234 (strain ANU240)
cttaacacatgcaagtcgagcgccccgcaaggggagcggcagacgggtgagtaacgcgtgggaatctacccttttctacggaataacgcagggaaacttgtgctaataccgtatgagcccttcgggggaaagatttatcgggaaaggatgagcccgcgttggattagctagttggtggggtaaaggcctaccaaggcgacgatccatagctggtctgagaggatgatcagccacattgggactgagacacggcccaaactcctacgggaggcagcagtggggaatattggacaatgggcgcaagcctgatccagccatgccgcgtgagtgatgaaggccctagggttgtaaagctctttcaccggtgaagataatgacggtaaccggagaagaagccccggctaacttcgtgccagcagccgcggtaatacgaagggggctagcgttgttcggaattactgggcgtaaagcgcacgtaggcggacatttaagtcaggggtgaaatcccggggctcaaccccggaactgcctttgatactgggtgtctagagtccggaagaggtgagtggaattccgagtgtagaggtgaaattcgtagatattcggaggaacaccagtggcgaaggcggctcactggtccggtactgacgctgaggtgcgaaagcgtggggagcaaacaggattagataccctggtagtccacgccgtaaacgatgaatgttagccgtcgggcagtttactgttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagcccttgacatcccggtcgcggatacgagagatcgtatccttcagttcggctggaccggagacaggtgctgcatggctgtcgtcagctcgtgtcgtgagatgttgggttaagtcccgcaacgagcgcaaccctcgcccttagttgccagcatttggttgggcactctaaggggactgccggtgataagccgagaggaaggtggggatgacgtcaagtcctcatggcccttacgggctgggctacacacgtgctacaatggtggtgacagtgggcagcgagaccgcgaggtcgagctaatctccaaaagccatctcagttcggattgcactctgcaactcgagtgcatgaagttggaatcgctagtaatcgcagatcagcatgctgcggtgaatacgttcccgggccttgtacacaccgcccgtcacaccatgggagttggttctacccgaaggtagtgcgctaaccgcaaggaggcagctaaccacggtagggtcagcgactggggtgaagtcgtaacaaggtagccg

SEQ ID NO: 2 - DNA, 487 bp, Rhizobium spp. NGR234 (ANU240 strain)
cgacgaagccggcccggtcgagacctcgtcgcgccgcgccatccaccaggcacgcgccggcctatgtcgagcagtcgacggaagcgcagatcctcgtcaccggcatcaaggtcgtcgacctgctggcgccttacgcaaagggcggcaagatcggcctcttcggcggcgcaggcgtcggcaagaccgttctgatcatggaactgatcaacaacgtcgcaaaggcccacggcggctactcggttttcgcgggcgtcggtgaacgcacgcgcgaaggcaacgacctctaccacgaaatgatcgaatccggcgtgaacaagcacggcggcggcgaaggctccaaggcagccctggtttacggccagatgaacgaaccgccgggcgcccgcgcccgcgtcgcactcaccggtctgacgatcgccgagcatttccgtgacgaaggtcaggacgttctcttcttcgtcgacaacatcttccgcttcacccaggc

SEQ ID NO: 3 - DNA, 474 bp, Rhizobium sp. NGR234 (ANU240)
tcggcaagggatcgatcatgaagctcggctcgaaggacagcgtaatcgagatcgaaactgtttcgaccggctcgctcggcctcgatatcgcgctcggcatcggcggtctgccgaaagggcgcatcattgaaatctatggtccggaaagctcgggcaagacgacgctggcgctgcaaaccattgccgaagcgcaaaaaaagggcggcatctgcggtttcgtcgacgccgaacatgcgctcgatccggtttatgcgcgcaaactgggcgtcgacctcgaaaacctgctgatctcgcagcccgataccggtgaacaggcgctcgaaatcaccgacacgctggttcgctccggtgcaatcgacgtgctcgtcgtcgactcggttgcagcgctcgtgccgcgcgccgaaatcgaaggcgagatgggcgacagcctgccgggcatgcaggcccgcctgatgagccaggcgctgcgcaaat

SEQ ID NO: 4 - DNA, 1395 bp, S. meliloti 1021
ggcttaacacatgcaagtcgagcgccccgcaaggggagcggcagacgggtgagtaacgcgtgggaatctacccttttctacggaataacgcagggaaacttgtgctaataccgtatgagcccttcgggggaaagatttatcgggaaaggatgagcccgcgttggattagctagttggtggggtaaaggcctaccaaggcgacgatccatagctggtctgagaggatgatcagccacattgggactgagacacggcccaaactcctacgggaggcagcagtggggaatattggacaatgggcgcaagcctgatccagccatgccgcgtgagtgatgaaggccctagggttgtaaagctctttcaccggtgaagataatgacggtaaccggagaagaagccccggctaacttcgtgccagcagccgcggtaatacgaagggggctagcgttgttcggaattactgggcgtaaagcgcacgtaggcggattgttaagtgaggggtgaaatcccagggctcaaccctggaactgcctttcatactggcaatctagagtccagaagaggtgagtggaattccgagtgtagaggtgaaattcgtagatattcggaggaacaccagtggcgaaggcggctcactggtctggaactgacgctgaggtgcgaaagcgtggggagcaaacaggattagataccctggtagtccacgccgtaaacgatgaatgttagccgtcgggcagtttactgttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagcccttgacatcccgatcgcggatacgagagatcgtatccttcagttcggctggatcggagacaggtgctgcatggctgtcgtcagctcgtgtcgtgagatgttgggttaagtcccgcaacgagcgcaaccctcgcccttagttgccagcattcagttgggcactctaaggggactgccggtgataagccgagaggaaggtggggatgacgtcaagtcctcatggcccttacgggctgggctacacacgtgctacaatggtggtgacagtgggcagcgagaccgcgaggtcgagctaatctccaaaagccatctcagttcggattgcactctgcaactcgagtgcatgaagttggaatcgctagtaatcgcagatcagcatgctgcggtgaatacgttcccgggccttgtacacaccgcccgtcacaccatgggagttggttctacccgaaggtagtgcgctaaccgcaaggaggcagctaaccacggtagggtcagcgactggggtgaagtc

SEQ ID NO: 5 - DNA, 362 bp, S. meliloti 1021
gctccctatgcgaagggcggcaagatcggcctcttcggcggcgcgggcgtcggcaagaccgtgctgatcatggaactgatcaacaacgtcgccaaggcgcacggcggttactccgtcttcgcaggcgtcggtgaacggacccgcgaaggcaacgacctctatcacgaaatgatcgagtccggcgtgaacaagcatggcggcggcgaaggttccaaggccgcgctcgtctacggccagatgaacgagccgccgggcgcccgcgcacgcgtcgcgctgaccggcctgacggtcgccgaacagttccgtgacgaaggccaggacgttctcttcttcgtcgacaacatcttccgcttcacccaggc

SEQ ID NO: 6 - DNA, 462 bp, S.meliloti 1021
gttcggcaagggatcgatcatgaagctcggagcgaaggacagcgtagttgagatcgaaaccgtctccaccggttcgctcggcctcgatatcgcgctcggcatcggcggcctgccgaagggccgtatcatcgagatctatggtccggaaagctcgggcaagacgacgctggcgctgcagaccattgccgaggcacagaagaagggcggcatctgcggcttcgtcgatgccgagcatgcactcgatccggtctatgcacgaaagctcggggtcgatctcgaaaatctcctgatttcgcagcctgatacgggcgagcaggcgctggaaatcaccgatacgctggtccgttcgggtgcgatcgacattctcgtcatcgattcggtggccgcactcgtgccgcgcgccgaaatcgagggcgagatgggcgacagtctgccgggcatgcaggcgcgcctcatgagcca

SEQ ID NO: 7 - DNA, 1349 bp, M. loti MAFF303099
cgggtgagtaacgcgtgggaatctacccatctctacggaacaactccgggaaactggagctaataccgtatacgtccttttggagaaagatttatcggagatggatgagcccgcgttggattagctagttggtggggtaatggcctaccaaggcgacgatccatagctggtctgagaggatgatcagccacactgggactgagacacggcccagactcctacgggaggcagcagtggggaatattggacaatgggcgcaagcctgatccagccatgccgcgtgagtgatgaaggccctagggttgtaaagctctttcaacggtgaagataatgacggtaaccgtagaagaagccccggctaacttcgtgccagcagccgcggtaatacgaagggggctagcgttgttcggaattactgggcgtaaagcgcacgtaggcggatacttaagtcaggggtgaaatcccggggctcaaccccggaactgcctttgatactgggtatctcgagtccggaagaggtgagtggaattccgagtgtagaggtgaaattcgtagatattcggaggaacaccagtggcgaaggcggctcactggtccggtactgacgctgaggtgcgaaagcgtggggagcaaacaggattagataccctggagtccacgccgtaaacgatggaagctagccgttggcaagtttacttgtcggtggcgcagctaacgcattaagcttcccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagagatggataccttcagttcggctggaccggtgacaggtgctgcatggctgtcgtcagctcgtgtcgtgagatgttgggttaagtcccgcaacgagcgcaaccctcgcccttagttgccagcattcagttgggcactctaaggggactgccggtgataagccgagaggaaggtggggatgacgtcaagtcctcatggcccttacgggctgggctacacacgtgctacaatggtggtgacagtgggcagcgagaccgcgaggtcgagctaatctccaaaagccatctcagttcggattgcactctgcaactcgagtgcatgaagttggaatcgctagtaatcgcggatcagcatgccgcggtgaatacgttcccgggccttgtacacaccgcccgtcacaccatgggagttggttttacccgaaggcgctgtgctaaccgcaaggaggcaggcgaccacggtagggtcagcgactggggtgaagtc

SEQ ID NO: 8 - DNA, 510 bp, M. loti MAFF303099
ttatcggcgagccggtcgacgaagagggcccggtcgatgcgatcgagatgcgctccatccaccagccggctccgacctatgtcgagcagtcgacggaagcgcagatcctgatcaccggcatcaaggtgctcgacctgctggcgccttacgccaagggcggcaagatcggcctgttcggcggcgccggcgtcggcaagaccgtgctgatccaggaactgatcaacaacatcgccaaggcacacggcggctattcggtgttcgccggcgtcggtgagcgcacccgcgagggcaacgatctctatcacgagttcatcgaatccggcgtcaacaagaagggcggcggcgaaggctccaaggcggctctcgtgtacggccagatgaacgagccgccgggcgcgcgcgcccgtgtcggcctgaccggcctgacggtggctgaatatttccgcgaccagggccaggacgtgctgttcttcgtcgacaacatcttccgcttcacgcaggctggttcgg

SEQ ID NO: 9 - DNA, 438 bp, M. loti MAFF 303099
ggcgaacgagcaggtcgtcgagatcgaaaccgttgccgaccggctcgctcggcctcgacatcgcgctcggcgtcggtggcctgccgcgcggccgcatcatcgagatctacggaccggaaagctcgggcaagacgacgctggccctccacacggtggccgaagcccagaagaagggcggcatctgcgccttcgtcgacgccgaacacgcgctcgatccggtctatgcccgcaagctcggcgtcgaccttgaaaacctgctgatctcgcagcccgataccggcgagcaggcgctggagatctgcgacacgctggtgcgttccggcgccatcgacgtgctggtggtcgattcggtcgcggcactgacgccgcgcgccgaaatcgagggcgagatgggcgattcgctgcccggcctgcaggctcgactgatgagccaggcgc

SEQ ID NO: 10 - DNA, 1368 bp, P. myrsinacearum Cambia isolate
cttaacacatgcaagtcgagcgccccgcaaggggagcggcagacgggtgagtaacgcgtgggaatctacccatctctacggaataacgcatggaaacgtgtgctaataccgtatacgtccttcgggagaaagatttatcggagatggatgagcccgcgttggattagctagttggtggggtaaaggcctaccaaggcgacgatccatagctggtctgagaggatgatcagccacactgggactgagacacggcccagactcctacgggaggcagcagtggggaatattggacaatgggcgcaagcctgatccagccatgccgcgtgagtgatgaaggccctagggttgtaaagctctttcaccggtgaagataatgacggtaaccggagaagaagccccggctaacttcgtgccagcagccgcggtaatacgaagggggctagcgttgttcggatttactgggcgtaaagcgcacgtaggcggactattaagtcaggggtgaaatcccggggctcaaccccggaactgcctttgatactggtagtcttgagttcgagagaggtgagtggaattccgagtgtagaggtgaaattcgtagatattcggaggaacaccagtggcgaaggcggctcactggctcgatactgacgctgaggtgcgaaagcgtggggagcaaacaggattagataccctggtagtccacgccgtaaactatgagagctagccgtcgggcagtatactgttcggtggcgcagcaaacgcattaagctctccgcctggggagtacggtcgcaagattaaaactcaaaggaattgacgggggcccgcacaagcggtggagcatgtggtttaattcgaagcaacgcgcagaaccttaccagcccttgacatcccgatcgcggttaccagagatggtttccttcagttaggctggatcggtgacaggtgctgcatggctgtcgtcagctcgtgtcgtgagatgttgggttaagtcccgcaacgagcgcaaccctcgcccttagttgccatcattcagttgggcactctaaggggactgccggtgataagccgagaggaaggtggggatgacgtcaagtcctcatggcccttacgggctgggctacacacgtgctacaatggtggtgacagtgggcagcgagaccgcgaggtcgagctaatctccaaaagccatctcagttcggattgcactctgcaactcgagtgcatgaagttggaatcgctagtaatcgtggatcagaatgccacggtgaatacgttcccgggccttgtacacaccgcccgtcacaccatgggagttggttttacccgaaggtgctgtgctaaccgcaaggaggcaggcaaccacgg

SEQ ID NO: 11 - DNA, 483 bp, P. myrsinacearum Cambia isolate
cgaagcaggcccgatcaagacgaagctgacccgcgctatccaccagccagctccggaatatgtcgagcagtcgacggaagcccagatcctggtaaccggcatcaaggttatcgatctgctggcgccttatgcgcgcggcggcaagatcggcctcttcggcggtgccggtgttggcaagacggttctgatcatggaactgatcaacaacgtggccaaggcgcacggcggctattcggtatttgccggtgtgggtgaacgtacccgcgaaggcaacgacctttaccacgaaatgatcgagtccggcgtgaacaaggccggcggcggtgaaggctccaaggccgcactggtttacggtcagatgaacgagccgccaggggcacgtgcccgcgttgcgctctccggtctgacggttgcggaacatttccgcgatgaaggccaggacgttctgttcttcgtggacaatattttccgcttcacgcaggc

SEQ ID NO: 12 - DNA, 1382 bp, B. japonicum USDA110
tagcaagtcgagcgggcgtagcaatacgtcagcggcagacgggtgagtaacgcgtgggaacgtaccttttggttcggaacaacacagggaaacttgtgctaataccggataagcccttacggggaaagatttatcgccgaaagatcggcccgcgtctgattagctagttggtagggtaacggcctaccaaggcgacgatcagtagctggtctgagaggatgatcagccacattgggactgagacacggcccaaactcctacgggaggcagcagtggggaatattggacaatgggggcaaccctgatccagccatgccgcgtgagtgatgaaggccctagggttgtaaagctcttttgtgcgggaagataatgacggtaccgcaagaataagccccggctaacttcgtgccagcagccgcggtaatacgaagggggctagcgttgctcggaatcactgggcgtaaagggtgcgtaggcgggtctttaagtcaggggtgaaatcctggagctcaactccagaactgcctttgatactgaagatcttgagttcgggagaggtgagtggaactgcgagtgtagaggtgaaattcgtagatattcgcaagaacaccagtggcgaaggcggctcactggcccgatactgacgctgaggcacgaaagcgtggggagcaaacaggattagataccctggtagtccacgccgtaaacgatgaatgccagccgttagtgggtttactcactagtggcgcagctaacgctttaagcattccgcctggggagtacggtcgcaagattaaaactcaaaggaattgacgggggcccgcacaagcggtggagcatgtggtttaattcgacgcaacgcgcagaaccttaccagcccttgacatgtccaggaccggtcgcagagatgtgaccttctcttcggagcctggaacacaggtgctgcatggctgtcgtcagctcgtgtcgtgagatgttgggttaagtcccgcaacgagcgcaacccccgtccttagttgctaccatttagttgagcactctaaggagactgccggtgataagccgcgaggaaggtggggatgacgtcaagtcctcatggcccttacgggctgggctacacacgtgctacaatggcggtgacaatgggatgctaaggggcgacccttcgcaaatctcaaaaagccgtctcagttcggattgggctctgcaactcgagcccatgaagttggaatcgctagtaatcgtggatcagcacgccacggtgaatacgttcccgggccttgtacacaccgcccgtcacaccatgggagttggctttacctgaagacggtgcgctaaccagcaatggaggcagccggccacggtagggtcagcgactgggt

SEQ ID NO: 13 - DNA, 504 bp, B. japonicum USDA110
cgatcgacgaagccggcccggtcaagtcggaaggcctgcgcgccatccaccaggaagcgccgacctacaccgaccagtccaccgaagctgaaattctcgtcaccggcatcaaggtcgtcgatctcctggctccctatgcgaagggcggcaagatcggcctgttcggcggcgccggcgtcggcaagaccgtgctgattcaggagctgatcaacaacgtcgcgaaggcgcacggtggttactccgtgttcgccggcgtcggcgagcgtacccgcgagggcaacgacctctatcacgagttcatcgagtccaaggtcaacgccgatccgcacaatccggatccgagcgtgaagtcgaagtgcgcgctggtgttcggccagatgaacgagccgccgggcgcccgcgcccgcgtcgcgctcaccggtctgaccatcgcggaagacttccgcgacaagggccaggacgtgctgttcttcgtcgacaacatcttccgcttcacccaggccg

SEQ ID NO: 14 - DNA, 438 bp, B. japonicum USDA110
cccggcagcgcatcgcccatctcgccctcgagttcggccttcggcaccagcgccgcgaccgaatcgaccaccagaacgtccaccgcacccgagcgcaccagcgtgtcgcagatttccagcgcctgctcgccggtgtccggctgcgaaatcagcagctcgtcgatattgaccccgagcttgcgtgcatagaccgggtcgagcgcgtgctcggcgtcgatgaaggcacagatgccgcccttcttctgcgcttccgccaccgtgtgcagcgccagcgtggtcttgcccgaggattccggcccgtagatttccacgacgcgccccttgggcagaccgccgatccccagtgcaatgtcgagcccgagagaaccggaggacaccgcctcgacatccatcgaccggtcgttcttgccgagcttcatcaccgagcccttgccgaac

SEQ ID NO: 15 - DNA, 1433 bp, A. tumefaciens EHA105
caagtcgaacgccccgcaaggggagtggcagacgggtgagtaacgcgtgggaatctacccatctctgcggaatagctctgggaaactggaattaataccgcatacgccctacgggggaaagatttatcggggatggatgagcccgcgttggattagctagttggtggggtaaaggcctaccaaggcgacgatccatagctggtctgagaggatgatcagccacattgggactgagacacggcccaaactcctacgggaggcagcagtggggaatattggacaatgggcgcaagcctgatccagccatgccgcgtgagtgatgaaggccttagggttgtaaagctctttcaccgatgaagataatgacggtagtcggagaagaagccccggctaacttcgtgccagcagccgcggtaatacgaagggggctagcgttgttcggaattactgggcgtaaagcgcacgtaggcggatatttaagtcaggggtgaaatcccgcagctcaactgcggaactgcctttgatactgggtatcttgagtatggaagaggtaagtggaattccgagtgtagaggtgaaattcgtagatattcggaggaacaccagtggcgaaggcggcttactggtccattactgacgctgaggtgcgaaagcgtggggagcaaacaggattagataccctggtagtccacgccgtaaacgatgaatgttagccgtcgggcagtatactgttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagctcttgacattcggggtatgggcattggagacgatgtccttcagttaggctggccccagaacaggtgctgcatggctgtcgtcagctcgtgtcgtgagatgttgggttaagtcccgcaacgagcgcaaccctcgcccttagttgccagcatttagttgggcactctaaggggactgccggtgataagccgagaggaaggtggggatgacgtcaagtcctcatggcccttacgggctgggctacacacgtgctacaatggtggtgacagtgggcagcgagacagcgatgtcgagctaatctccaaaagccatctcagttcggattgcactctgcaactcgagtgcatgaagttggaatcgctagtaatcgcagatcagcatgctgcggtgaatacgttcccgggccttgtacacaccgcccgtcacaccatgggagttggttttacccgaaggtagtgcgctaaccgcaaggaggcagctaaccacggtagggtcagcgactggggtgaagtcgtaacaaggtagccgtaggggaacctgcggctggatcacctcctttctaag

SEQ ID NO: 16 - DNA, 485 bp, A. tumefaciens EHA105
gacgaagccggtccgatcgtaacggccaagaagcgcgccatccaccaggacgcaccgtcttacgtcgagcagtcgacggaaggccagatcctcgtcaccggcatcaaggtcgtcgaccttctcgctccttacgccaagggcggcaagatcggcctcttcggcggcgccggcgtgggcaagacggttctcatcatggaactgatcaacaacgtcgccaaggcgcatggtggttactcggtattcgccggcgtgggtgagcgtacccgcgaaggcaacgacctttatcacgaaatgatcgagtcgaacgtcaacaagctcggtggcggcgaaggctccaaggctgcgctcgtgtacggccagatgaacgaaccgccgggcgcccgcgctcgcgtcgctctgaccggtctgacgatcgctgaaaacttccgtgatgaaggccaggacgttctgttcttcgtggacaacatcttccgcttcacgcaggc

SEQ ID NO: 17 - DNA, 400 bp, A. tumefaciens EHA105
ggcaagggatcgatcatgaagctcggttccaatgaaaatgtggttgaagtggaaaccgtttcgacgggctcgctcagcctggatatcgcgctcggcatcggcggcttgccgaaggggcgtatcattgagatttacggcccggaaagctccggtaaaacgacgctggcgctgcagacgatcgcggaagcccagaagaagggcggcatctgcgccttcgtggacgccgaacacgcgctcgatccggtttatgcccgcaagctcggtgtggatttgcagagccttctgatctcgcagcccgataccggcgagcaggcgcttgagatcaccgatacgctggtgcgttcgggcgctgtggacgttcttgtcatcgattcggttgcggccttgacgccgcgggcgg

SEQ ID NO: 18 - DNA, 18 bp, artificial
  synthetic DNA
cacgtaggcggatcgatc

SEQ ID NO: 19 - DNA, 20 bp, artificial
  synthetic DNA
ttagctcacactcgcgtgct

SEQ ID NO: 20 - DNA, 23 bp, artificial
  synthetic DNA
ggcttaacacatgcaagtcgaac

SEQ ID NO: 21 - DNA, 19 bp, artificial
  synthetic DNA
cggggcttcttctccgact

SEQ ID NO: 22 - DNA, 23 bp, artificial
  synthetic DNA
gaatagctctgggaaactggaat

SEQ ID NO: 23 - DNA, 20 bp, artificial
  synthetic DNA
caggctcaaaccgcatttcc

SEQ ID NO: 24 - DNA, 21 bp, artificial
  synthetic DNA
gtaagtccagcctctttctca

SEQ ID NO: 25 - DNA, 20 bp, artificial
  synthetic DNA
gtgcttcggatcgacgaaac

SEQ ID NO: 26 - DNA, 20 bp, artificial
  synthetic DNA
ggagaatgggagtgacctga

SEQ ID NO: 27 - DNA, 20 bp, artificial
  synthetic DNA
cgctaagccgtttagtacga

SEQ ID NO: 28 - DNA, 24 bp, artificial
  synthetic DNA
cccctcaccaaatattgagtgtag

SEQ ID NO: 29 - DNA, 20 bp, artificial
  synthetic DNA
caggtgcgacaatctatcga

SEQ ID NO: 30 - DNA, 20 bp, artificial
  synthetic DNA
agccgtttctgtaatgaagg

SEQ ID NO: 31 - DNA, 20 bp, artificial
  synthetic DNA
tgaccttggccagggaattg

SEQ ID NO: 32 - DNA, 20 bp, artificial
  synthetic DNA
tcctgtcattggcgtcagtt

SEQ ID NO: 33 - DNA, 20 bp, artificial
  synthetic DNA
tgtgctaataccgtatgagc

SEQ ID NO: 34 - DNA, 20 bp, artificial
  synthetic DNA
cagccgaactgaaggatacg

SEQ ID NO: 35 - DNA, 23 bp, artificial
  synthetic DNA
gccagaaatgttcatgtcgcaca

SEQ ID NO: 36 - DNA, 21 bp, artificial
  synthetic DNA
aatgggttgcggaagttcggt

SEQ ID NO: 37 - DNA, 20 bp, artificial
  synthetic DNA
gacaggatcctccacgctca

SEQ ID NO: 38 - DNA, 19 bp, artificial
  synthetic DNA
cgccaggtcgttcggttgg

SEQ ID NO: 39 - DNA, 20 bp, artificial
  synthetic DNA
gctcatagggcgaggataca

SEQ ID NO: 40 - DNA, 20 bp, artificial
  synthetic DNA
acggcgcgaatccaatccaa

SEQ ID NO: 41 - DNA, 17 bp, artificial
  synthetic DNA
caggaaacagctatgac

SEQ ID NO: 42 - DNA, 16 bp, artificial
  synthetic DNA
gtaaaacgacggccag

SEQ ID NO: 43 - DNA, 20 bp, artificial
  synthetic DNA
taagcgtcccatcgagatcg

SEQ ID NO: 44 - DNA, 20 bp, artificial
  synthetic DNA
gcatctcccgccgtgcacag

SEQ ID NO: 45 - DNA, 22 bp, artificial
  synthetic DNA
gatgcctccgctcgaagtagcg

SEQ ID NO: 46 - DNA, 17 bp, artificial
  synthetic DNA
ctggcacgacaggtttc

SEQ ID NO: 47 - DNA, 21 bp, artificial
  synthetic DNA
caggcttaacacatgcaagtc

SEQ ID NO: 48 - DNA, 20 bp, artificial
  synthetic DNA
accagggtatctaatcctgt

SEQ ID NO: 49 - DNA, 19 bp, artificial
  synthetic DNA
gaacaccagtggcgaaggc

SEQ ID NO: 50 - DNA, 20 bp, artificial
  synthetic DNA
cggctaccttgttacgactt

SEQ ID NO: 51 - DNA, 20 bp, artificial
  synthetic DNA
atcggcgagccggtcgacga

SEQ ID NO: 52 - DNA, 23 bp, artificial
  synthetic DNA
gccgacacttccgaaccngcctg

SEQ ID NO: 53 - DNA, 23 bp, artificial
  synthetic DNA
atcgagcggtcgttcggcaaggg

SEQ ID NO: 54 - DNA, 20 bp, artificial
  synthetic DNA
ttgcgcagcgcctggctcat

SEQ ID NO: 55 - DNA, 20 bp, artificial
  synthetic DNA
cccatctctacggaacaact

SEQ ID NO: 56 - DNA, 20 bp, artificial
  synthetic DNA
actcacctcttccggactcg

SEQ ID NO: 57 - DNA, 20 bp, artificial
  synthetic DNA
gacggccgagccaaggacga

SEQ ID NO: 58 - DNA, 19 bp, artificial
  synthetic DNA
cacatggcaagcctcctca

SEQ ID NO: 59 - DNA, 22 bp, artificial
  synthetic DNA
gatgctggaaagcttcacaagt

SEQ ID NO: 60 - DNA, 20 bp, artificial
  synthetic DNA
ctggtagtcttgagttcgag

SEQ ID NO: 61 - DNA, 20 bp, artificial
  synthetic DNA
ccagcctaactgaaggaaac

SEQ ID NO: 62 - DNA, 20 bp, artificial
  synthetic DNA
ctggctgcgtctcaagattc

SEQ ID NO: 63 - DNA, 20 bp, artificial
  synthetic DNA
cctttgccttcttcgcctgc

SEQ ID NO: 64 - DNA, 18 bp, artificial
  synthetic DNA
gggcgtagcaatacgtca

SEQ ID NO: 65 - DNA, 20 bp, artificial
  synthetic DNA
cttcgccactggtgttcttg

SEQ ID NO: 66 - DNA, 28 bp, artificial
  synthetic DNA
ataagcttctctacggcgatcgatgtca

SEQ ID NO: 67 - DNA, 28 bp, artificial
  synthetic DNA
atctgcagtgctcgaggtcgctcgaagt

SEQ ID NO: 68 - DNA, 28 bp, artificial
  synthetic DNA
atggatccggtcttgaaagcttggctca

SEQ ID NO: 69 - DNA, 29 bp, artificial
  synthetic DNA
atggatcctgccgtggtctcgtgttctgg

SEQ ID NO: 70 - DNA, 28 bp, artificial
  synthetic DNA
atggatccgagcagggagaggacaacca

SEQ ID NO: 71 - DNA, 28 bp, artificial
  synthetic DNA
atggatcctcgggtcctgaaagatcatc

SEQ ID NO: 72 - DNA, 36 bp, artificial
  synthetic DNA
ggatcctctagactggaaggcagtacaccttgatag

SEQ ID NO: 73 - DNA, 35 bp, artificial
  synthetic DNA
ggatcctctagattcctgcatttgcctgtttccag

SEQ ID NO: 74 - DNA, 19 bp, artificial
  synthetic DNA
agctgcggaagaagctcgt

SEQ ID NO: 75 - DNA, 20 bp, artificial
  synthetic DNA
taagcgtcccatcgagatcg

SEQ ID NO: 76 - DNA, 26 bp, artificial
  synthetic DNA
atgcatgatatatctcccaatttgtg

SEQ ID NO: 77 - DNA, 36 bp, artificial
  synthetic DNA
ccgcggatgacagagcgttgctgcctgtgatcaatt

SEQ ID NO: 78 - DNA, 26 bp, artificial
  synthetic DNA
ccgcggcatgatatatctcccaattt

SEQ ID NO: 79 - DNA, 20 bp, artificial
  synthetic DNA
tacggcgagttctgttaggt

SEQ ID NO: 80 - DNA, 20 bp, artificial
  synthetic DNA
agctggctgacgaacctgcg

SEQ ID NO: 81 - DNA, 20 bp, artificial
  synthetic DNA
ggcgtccttggaacgatgct

SEQ ID NO: 82 - DNA, 19 bp, artificial
  synthetic DNA
actcaccgcgacgtctgtc

SEQ ID NO: 83 - DNA, 18 bp, artificial
  synthetic DNA
gcgcgtctgctgctccat